(19) United States
(12) Patent Application Publication
(10) Pub. No.: US 2024/0408188 A1
(43) Pub. Date: Dec. 12, 2024

(54) NANOEMULSION ADJUVANT COMPOSITIONS FOR HUMAN PAPILLOMAVIRUS VACCINES

(71) Applicant: Merck Sharp & Dohme LLC, Rahway, NJ (US)

(72) Inventors: Patrick L. Ahl, Yardley, PA (US); William J. Smith, Harleysville, PA (US); Randal J. Soukup, Hatfield, PA (US); Nicole Lea Sullivan, Hatboro, PA (US); John Gaspar, Boston, MA (US); Julie M. Skinner, Phoenixville, PA (US)

(73) Assignee: Merck Sharp & Dohme LLC, Rahway, NJ (US)

(21) Appl. No.: 18/735,616

(22) Filed: Jun. 6, 2024

Related U.S. Application Data
(60) Provisional application No. 63/507,269, filed on Jun. 9, 2023.

(51) Int. Cl. A61K 39/12; A61K 39/00; C12N 7/00

(52) U.S. Cl. CPC A61K 39/12; A61K 2039/55566; C12N 7/00; C12N 2710/20023; C12N 2710/20031

(57) ABSTRACT

The present disclosure provides, among other things, a vaccine composition that includes a squalene nanoemulsion (SNE) adjuvant and HPV virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

### CROSS-REFERENCE TO RELATED APPLICATIONS

This application claims the benefit of priority of U.S. Provisional Application No. 63/507,269, filed Jun. 9, 2023, the disclosure of which is incorporated herein by its entirety.

### FIELD OF THE INVENTION

The invention relates generally to the prevention of human papillomavirus (HPV) disease. More specifically, the invention relates to a composition comprising HPV virus-like particles (VLPs) and a squalene nanoemulsion (SNE) adjuvant, which can be administered as a vaccine. The SNE adjuvant comprises surfactants and/or terpenes and/or terpanoid-based oils and/or cationic lipids or mixtures thereof. Further provided are methods of using the disclosed compositions and formulations.

### BACKGROUND

Human papillomaviruses (HPVs) are small, double-stranded DNA viruses that infect the skin and internal squamous mucosal epithelia of men and women. HPVs are classified based on their carcinogenic properties. HPVs include major (L1) and minor (L2) capsid proteins. Over 200 distinct HPV genotypes have been identified (Li et al., “Rational design of a triple-type human papillomavirus vaccine by compromising viral-type specificity,” Nature, 9:5360 (2018)), many of which have been associated with pathologies ranging from benign proliferative warts to malignant carcinomas of the cervix (for review, see McMurray et al., Int. J. Exp. Pathol. 82 (1): 15-33 (2001)). Those HPV types labeled as “high-risk” include 16, 18, 31, 33, 35, 39, 45, 51, 52, 56, 58, 68, and 59. (Chan et al., “Human Papillomavirus Infection and Cervical Cancer: Epidemiology, Screening, and Vaccination-Review of Current Perspectives,” Journal of Oncology, vol. 2019, Article ID 3257939, 2019.)

HPV is the primary etiological agent in cervical cancer, one of the most common cancer types in women, as well as squamous cell carcinomas of the anus, tonsil, tongue, vulva, vagina, and penis. HPV16 and HPV18 are well known as the most virulent of the high-risk HPV types as they cause approximately 70% of all invasive cervical cancer in the world.

Papillomaviruses are small (50-60 run), nonenveloped, icosahedral DNA viruses that encode up to eight early (E1-E7) and two late (L1-L2) genes. The L1 protein is the major capsid protein and has a molecular weight of 55-60 kDa. Expression of the L1 protein or a combination of the L1 and L2 proteins in yeast, insect cells, mammalian cells or bacteria leads to self-assembly of virus-like particles (VLPs) (for review, see Schiller and Roden, in Papillomavirus Reviews: Current Research on Papillomaviruses; Lacey, ed. Leeds, UK: Leeds Medical Information, pp 101-12 (1996)).

VLPs are morphologically similar to authentic virions and are capable of inducing high titers of neutralizing antibodies upon administration into animals or humans. Because VLPs do not contain the potentially oncogenic viral genome, they present a safe alternative to the use of live virus in HPV vaccine development (for review, see Schiller and Hidesheim, J Clin. Virol. 19:67-74 (2000)).

VLP-based vaccines have proven to be effective at inducing immune responses in human subjects vaccinated with bivalent HPV 16 and 18 (Harper et al. Lancet 364 (9447): 1757-65 (2004)), quadrivalent HPV 6, 11, 16, and 18 (Villa et al. Vaccine 24:5571-5583 (2006)) and multi-valent HPV 6, 11, 16, 18, 31, 33, 45, 52 and 58 VLP-based vaccines. Three marketed VLP-based vaccines against HPV are administered according to 2 or 3 dose regimens. CERVARIX® (GlaxoSmithKline Biologicals, Rixensart, Belgium), is a bivalent vaccine protective against HPV 16 and 18. GARDASIL® and GARDASIL®9 (Merck & Co., Inc., Rahway, NJ, USA) protect against two and seven additional HPV types, respectively, and prevent additional HPV-related anogenital diseases, including wart formation. The additional five high-risk strains in GARDASIL®9 compared to GARDASIL® increase protection from about 70% of anogenital malignancies to about 90%. (Id., M. Nygård, et al., “Evaluation of the long-term anti-human papillomavirus 6 (HPV6), 11, 16, and 18 immune responses generated by the quadrivalent HPV vaccine,” Clinical and Vaccine Immunology, vol. 22, no. 8, pp. 943-948, 2015.)

Though improving, worldwide HPV vaccination rates remain suboptimal. The worldwide coverage of HPV vaccination rates can be improved by reducing the number of healthcare practitioner visits required for the vaccination, increasing education on HPV disease prophylaxis, and alleviating the social stigma associated with vaccination.

Moreover, licensed HPV vaccines currently utilize aluminum containing derivatives as adjuvants to increase immunogenicity. Even though aluminum adjuvants increase immunogenic responses from baseline, it is unknown whether the immunogenic response is sufficient for higher valency HPV vaccines. Therefore, there is a need to identify other adjuvants that can provide increased immunogenicity for multivalent HPV vaccines over the current aluminum adjuvant.

### SUMMARY OF THE INVENTION

The invention provides composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 adsorbed on an aluminum adjuvant and a squalene nanoemulsion (SNE) adjuvant, wherein the SNE adjuvant comprises sorbitan trioleate (SPAN-85); polysorbate-20 (PS-20) or polysorbate-80 (PS-80); and squalene.

The invention further provides compositions comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 and a squalene nanoemulsion (SNE) adjuvant, wherein the SNE adjuvant comprises sorbitan trioleate (SPAN-85); polysorbate-20 (PS-20) or polysorbate-80 (PS-80); and squalene. In some embodiments, the composition also includes an aluminum adjuvant. In some embodiments, the VLPs are adsorbed on an aluminum adjuvant.

The invention further provides a method of inducing an immune response to a human papillomavirus (HPV) in a human patient comprising administering to the patient a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 and a squalene nanoemulsion (SNE) adjuvant, wherein the SNE adjuvant comprises sorbitan trioleate (SPAN-85); polysorbate-20 (PS-20) or polysorbate-80 (PS-80); and squalene, and an optionally an aluminum adjuvant.

The disclosure further provides, among other things, a method of preventing infection of or reducing the likelihood of infection of a human patient by a human papillomavirus (HPV) comprising administration to the patient the pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 adsorbed on an aluminum adjuvant and a squalene nanoemulsion (SNE) adjuvant, wherein the SNE adjuvant comprises sorbitan trioleate (SPAN-85); polysorbate-20 (PS-20) or polysorbate-80 (PS-80); and squalene.

The invention further provides the use of comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 adsorbed on an aluminum adjuvant and a squalene nanoemulsion (SNE) adjuvant, wherein the SNE adjuvant comprises sorbitan trioleate (SPAN-85); polysorbate-20 (PS-20) or polysorbate-80 (PS-80); and squalene for preventing infection of or reducing the likelihood of infection of a human patient by a human papillomavirus (HPV).

The invention also provides a kit comprising: (1) a human papillomavirus (HPV) vaccine; and (2) a squalene nanoemulsion (SNE) adjuvant, wherein the SNE adjuvant comprises sorbitan trioleate (SPAN-85); polysorbate-20 (PS-20) or polysorbate-80 (PS-80); and squalene, and an optionally an aluminum adjuvant.

### Definitions

As used throughout the specification and in the appended claims, the singular forms “a,” “an,” and “the” include the plural reference unless the context clearly dictates otherwise.

As used throughout the specification and appended claims, the following abbreviations and definitions apply:
CLA ((13Z,16Z)-N,N-dimethyl-3-nonyldocosa-13,16-dien-1-amine)
HPV human papillomavirus
ID intradermal
IM intramuscular
ME microemulsion
MNS microfluidic nanoemulsion self-assembly
Mw molecular weight
NE nanoemulsion
NMWCO nominal molecular weight cut off
PHE pre-homogenized emulsion
PS-20 polysorbate-20
PS-80 polysorbate-80
SNE squalene nanoemulsion
SPAN-85 sorbitan-trioleate
VPL(s) virus-like particle(s) (VLPs)
w/v weight per volume

As used throughout the specification and appended claims, the following definitions and abbreviations apply:

AAHS: As used herein, the term “AAHS” refers to an amorphous aluminum hydroxyphosphate sulfate adjuvant.

About: As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Adjuvant: As used herein, the term “adjuvant” refers to a composition or compound that is capable of enhancing the immune response against an antigen of interest. Adjuvants are substances or combinations of substances that are used in conjunction with a vaccine antigen to enhance (e.g., increase, accelerate, prolong and/or possibly target) the specific immune response to the vaccine antigen or modulate to a different type (e.g., switch a Th1 immune response to a Th2 response, or a humoral response to a cytotoxic T cell response) in order to enhance the clinical effectiveness of the vaccine. In some embodiments, the adjuvant modifies (Th1/Th2) the immune response. In some embodiments, the adjuvant boosts the strength and longevity of the immune response. In some embodiments, the adjuvant broadens the immune response to a concomitantly administered antigen. In some embodiments, the adjuvant is capable of inducing strong antibody and T cell responses. In some embodiments, the adjuvant is capable of increasing the polyclonal ability of the induced antibodies. In some embodiments, the adjuvant is used to decrease the amount of antigen necessary to provoke the desired immune response and provide protection against the disease. In some embodiments, the adjuvant is used to decrease the number of injections needed in a clinical regimen to induce a durable immune response and provide protection against the disease. Adjuvant containing formulations described herein may demonstrate enhancements in humoral and/or cellular immunogenicity of vaccine antigens, for example, subunit vaccine antigens. Adjuvants of the invention are not used to deliver antigens, antibodies, active pharmaceutical ingredients (APIs), or VLPs.

Administration: As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), car, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a VLP, a small molecule, polypeptide (e.g., a protein), polynucleotide (e.g., a DNA polynucleotide or an RNA polynucleotide), saccharide, lipid, or a combination or complex thereof. In some embodiments, the term “agent” can refer to a compound, molecule, or entity that includes a polymer, or a plurality thereof.

Alkenyl: As used herein, the term “alkenyl” refers to a straight chain, cyclic or branched unsaturated aliphatic hydrocarbon having the specified number of carbon atoms. In one embodiment, an alkenyl group contains from 8 to 24 carbon atoms (C₈-C₂₄ alkenyl). In one embodiment, an alkenyl group is linear. In another embodiment, an alkenyl group is branched. In another embodiment the alkenyl group is unsubstituted.

Alkyl: As used herein, the term “alkyl” refers to a straight chain, cyclic or branched saturated aliphatic hydrocarbon having the specified number of carbon atoms. In one embodiment, an alkyl group contains from 8 to 24 carbon atoms (C₈-C₂₄ alkyl). In one embodiment, an alkyl group is linear. In another embodiment, an alkyl group is branched. In another embodiment the alkyl group is unsubstituted.

Antibody: As used herein, the term “antibody” (or “Ab”) refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies.

Antigen: As used herein, the term “antigen” refers to any antigen that can generate one or more immune responses. The antigen may be a protein (including recombinant proteins), VLP, polypeptide, or peptide (including synthetic peptides). The antigen may be one that generates a humoral and/or CTL immune response.

API: As used herein, the term “API” refers to an active pharmaceutical ingredient, e.g., HPV VLPs, which is a component of the compositions or formulations disclosed herein that is biologically active (e.g., capable of inducing an appropriate immune response) and confers a therapeutic or prophylactic benefit to a person or animal in need thereof. As used herein, an API is a vaccine active ingredient.

Cationic lipid: As used herein, the term “cationic lipid” refers to a lipid species that carries a net positive charge at a selected pH, such as physiological pH. A cationic lipid may be utilized as an ingredient in a multi-component SNE adjuvant formulation. Those of skill in the art will appreciate that a cationic lipids can include, but are not limited to, those disclosed in US Patent Application Publication Nos. US2008/0085870, US2008/0057080, US2009/0263407, US2009/0285881, US2010/0055168, US2010/0055169, US2010/0063135, US2010/0076055, US2010/0099738, US2010/0104629, US2013/0017239, and US2016/0361411, International Application Publication Nos. WO2011/022460, WO2012/040184, WO2011/076807, WO2010/021865, WO 2009/132131, WO2010/042877, WO2010/146740, and WO2010/105209, and U.S. Pat. Nos. 5,208,036, 5,264,618, 5,279,833, 5,283,185, 6,890,557, and 9,669,097.

Co-administration: As used herein, the term “co-administration” or “co-administering” in relation to the SNE adjuvant and a pharmaceutical formulation (e.g., an HPV vaccine) refers to administration of an SNE adjuvant and a pharmaceutical formulation (e.g., an HPV vaccine) concurrently, i.e., simultaneously in time, or sequentially, i.e., administration of an HPV vaccine followed by administration of an SNE adjuvant (or vice versa). That is, after administration of the HPV vaccine (or SNE adjuvant), the SNE adjuvant (or HPV vaccine) can be administered substantially immediately after the HPV vaccine (or SNE adjuvant) or the SNE adjuvant (or the HPV vaccine) can be administered after an effective time period after the HPV vaccine (or SNE adjuvant); the effective time period is the amount of time period is generally within 1, 2, 3, 5, 10, 15, 20, 25, 30, 45, or 60 minutes.

Composition: As used herein, the term “composition” refers to a formulation containing an active pharmaceutical or biological ingredient (for example, a virus-like particle (VLP) of at least one type of human papillomavirus (HPV) and a SNE), along with one or more additional components. The term “composition” is used interchangeably with “pharmaceutical composition” and “formulation.” The compositions can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. Compositions are nontoxic to recipients at the dosages and concentrations employed.

Dose: As used herein, the term “dose” means a quantity of an agent, API, formulation, or pharmaceutical composition administered or recommended to be administered at a particular time.

HPV and PV: As used herein, the terms “HPV” and “PV” refer to human papillomavirus and papillomavirus, respectively.

Immunogenic: As used herein, the term “immunogenic” or “immunogenicity” refers to the ability of an antigen to provoke an immune response in a subject. The term “immunogenic composition” refers to the ability of an agent, API, formulation, or composition to provoke an immune response in a subject. The pneumococcal conjugate compositions of the invention are immunogenic compositions.

In need of treatment: Those “in need of treatment” include those previously exposed to or infected with human papillomavirus or papillomavirus, those who were previously vaccinated against human papillomavirus or papillomavirus, as well as those prone to have an infection or any person in which a reduction in the likelihood of infection is desired, e.g., the immunocompromised, the elderly, children, adults, or healthy individuals.

Lipid: As used herein, the term “lipid” refers to any of a group of organic compounds that are esters of fatty acids and are characterized by being insoluble in water or having low solubility in water but may be soluble in many organic solvents. Lipids can be divided in at least three classes: (1) “simple lipids,” which include, e.g., fats and oils as well as waxes; (2) “compound lipids,” which include, e.g., phospholipids and glycolipids; and (3) “derived lipids,” which include, e.g., steroids.

Patient: As used herein, the term “patient” refers to any human being that is to receive the HPV vaccines, or pharmaceutical compositions, described herein. As defined herein, “patient” includes those already infected with one or more types of HPV as well as those in which infection with one or more types of HPV is to be prevented.

Pharmaceutically acceptable: As used herein with respect to a carrier, diluent, or excipient of a pharmaceutical composition, the term “pharmaceutically acceptable” indicates that a carrier, diluent, or excipient must be compatible with the other ingredients of the composition and not rious to the recipient thereof.

Pharmaceutical composition: As used herein, the term “pharmaceutical composition,” refers to a composition containing an active pharmaceutical or biological ingredient, along with one or more additional components, e.g., a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Squalene nanoemulsion: As used herein, the terms “squalene nanoemulsion” or “SNE” refer to a formulation of emulsifiers and/or solubilizers and/or surfactants and/or lipids that have adjuvant properties in an HPV vaccine. Specifically, SNE refers to a SNE adjuvant formulation comprising (1) sorbitan trioleate (SPAN-85); (2) polysorbate-20 (PS-20); (3) squalene; and an optional (4) cationic lipid.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Surfactants: As used herein, the term “surfactants” refers to stabilizing ingredients in a multi-component SNE adjuvant formulation and include the polyoxyethylene sorbitan esters surfactants (commonly referred to as the Tweens, especially PS-20 and PS-80), copolymers of ethylene oxide (EO), propylene oxide (PO), and/or butylene oxide (BO), sold under the DOWFAX™ tradename, such as linear EO/PO block copolymers (poloxamers); octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy) polyethoxyethanol (IGEPAL CA-630/NP-40); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span-85, Tween-85 or [2-[(2R,3S,4R)-4-hydroxy-3-[(Z)-octadec-9-enoyl]oxyoxolan-2-yl]-2-[(Z)-octadec-9-enoyl]oxyethyl] (Z)-octadec-9-enoate) and sorbitan monolaurate. In an embodiment, surfactants are selected from sorbitan esters and poloxamers. In an embodiment, surfactants are selected from polysorbate-20 (PS-20) and polysorbate-80 (PS-80).

Terpenes: As used herein, the term “terpenes” refers to stabilizing ingredients in a multi-component SNE adjuvant formulation and include, but are not limited to: monoterpenes including geraniol, terpincol, limonene, myrcene, linalool and pinene; sesquiterpenes including humulene, farnesenes and farnesol; diterpenes including cafestol, kahweol, cembrene and taxadiene; triterpenes including squalene and squalane; tetraterpenes including acyclic lycopene, monocyclic gamma-carotene, bicyclic alpha- and beta-carotenes; polyterpines and norisopredoids. In an embodiment, the terpene is an oxidative degradant of terpene. In an embodiment, a terpene is squalene.

Therapeutically Effective Amount: As used herein, the term “therapeutically effective amount” (or “therapeutically effective dose”) refers to an amount of the active ingredient (e.g., therapeutic protein, vaccine, or antibody) sufficient to produce the desired therapeutic effect in a human or animal, e.g., the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given antibody or therapeutic protein or vaccine antigen.

Vaccine: As used herein, the term “vaccine” or “vaccine composition” refers to a substance or preparation used to stimulate the production of antibodies and provide immunity against one or several diseases, prepared from the causative agent of a disease, its products, or a synthetic substitute, treated to act as an antigen without inducing the disease. A vaccine composition may include at least one antigen or VLP in a pharmaceutically acceptable vehicle useful for inducing an immune response in a subject. The vaccine composition is administered by doses and techniques known to those skilled in the pharmaceutical or veterinary fields, taking into account factors such as the age, sex, weight, species, and condition of the recipient animal and the route of administration.

Valent: As used herein, the term “valent” refers to the presence of a specified number of antigens in a vaccine. For example, the terms bi-valent, bivalent, 2 valent, or 2-valent refer to two different antigens. Similarly, the terms quadrivalent, 4 valent, or 4-valent refer to four different antigens and the terms nonavalent, 9 valent or 9-valent refer to nine different antigens.

Virus-Like Particles: As used herein, the term “virus-like particles” or “VLPs” refers to agents that are morphologically similar to authentic virions or provide an arrayed display of an antigen and are capable of inducing high antibody neutralization ratings after administration in an animal. VLPs lack the viral genetic material of the authentic virions and are thus non-infectious.

### BRIEF DESCRIPTION OF THE DRAWINGS

FIG. 1: Select structures of cationic lipids: (13Z,16Z)-N,N-dimethyl-3-nonyldocosa-13,16-dien-1-amine (CLA); (6Z,9Z,26Z,29Z)-N,N-dimethylpentatriaconta-6,9,26,29-tetraen-18-amine (CLX); and N,N-dimethyl-1-((1S,2R)-2-octylcyclopropyl) heptadecan-8-amine (CLY). See Example 1.

FIG. 2: CLA-SNE components: (13Z,16Z)-N,N-dimethyl-3-nonyldocosa-13,16-dien-1-amine (CLA), SPAN-85, PS-20 and squalene. See Example 1.

FIG. 3: Characterization of CLA-SNE adjuvant bulk preparation utilizing static light scattering (SLS). See Example 2.

FIG. 4: Impact of the formulation process on the incorporation of CLA into an SNE. See Example 2.

FIGS. 5A-5D: Nanotracking analysis (NTA) of CLA-SNE and SNE formulations stored at 4° C. and 37° C. for 1 month (FIG. 5A: CLA-SNE [6 mg/mL CLA and 30 mg/mL squalene]; FIG. 5B: SNE [40 mg/mL squalene]; FIG. 5C: CLA-SNE [4 mg/mL CLA and 4 mg/mL squalene]; and FIG. 5D: SNE [8 mg/mL squalene]). See Example 3.

FIGS. 6A-6D: Dynamic light scattering (DLS) of CLA-SNE and SNE formulations stored at 4° C., 25° C. and 37° C. for 1 month (FIG. 6A: CLA-SNE [6 mg/mL CLA and 30 mg/mL squalene]; FIG. 6B: CLA-SNE [4 mg/mL CLA and 4 mg/mL squalene]; FIG. 6C: SNE [40 mg/mL squalene]; and FIG. 6D: SNE [8 mg/mL squalene]). See Example 3.

FIG. 7A: CLA concentration (mg/mL) as measured by UPLC-CAD for CLA-SNE and SNE formulations stored at 4° C., 25° C. and 37° C. for 1 month. See Example 4.

FIG. 7B: Squalene concentration (mg/mL) as measured by UPLC-CAD for CLA-SNE and SNE formulations stored at 4° C., 25° C. and 37° C. for 1 month. See Example 4.

FIG. 8A: The CLA/squalene (w/w) % after dialysis is plotted versus the “target” (w/w) % before self-assembly. The CLA/squalene w/w % ratios (X) were measured by reverse phase UPLC-CAD before and after self-assembly and nanoemulsion dialysis. See Example 5.

FIG. 8B: The measured intensity weighted Z-average DLS diameters of CLA-SNE nanoparticles after dialysis (X) is plotted versus the measured CLA/squalene (w/w) % after dialysis for each of MNS formulation. Sec Example 5.

FIG. 8C: The measured Zeta Potential of CLA-SNE squalene nanoparticles (X) after dialysis at pH 5.5 is plotted versus the measured CLA/squalene (w/w) % after dialysis for each of MNS prepared formulations. See Example 6.

FIG. 9: DLS Z-averages diameters of CLA-SNE samples formed and processed with aqueous phase (20 mM L-Histidine) of increasing pH values. See Example 6.

FIG. 10: Final [CLA] (mg/mL) of CLA-SNE samples formed and processed with aqueous phase (20 mM L-Histidine) of increasing pH values. See Example 6.

FIG. 11A: graphical representation of HPV VLP 16 antibody levels in Rhesus Macaques after two-doses of a 9vHPV vaccine combined with either a CLA-SNE or SNE adjuvant. Sec Example 7.

FIG. 11B: graphical representation of HPV VLP 18 antibody levels in Rhesus Macaques after two-doses of a 9vHPV vaccine combined with either a CLA-SNE or SNE adjuvant. Sec Example 7.

FIG. 12: graphical representation of individual HPV VLP antibody levels in Rhesus Macaques measured at 54 weeks after two-doses of a 9vHPV Vaccine combined with either a CLA-SNE or SNE adjuvant. See Example 7.

FIG. 13A: graphical representation of HPV VLP 16 antibody levels in Rhesus Macaques after two-doses of a 9vHPV Vaccine combined with either a CLA-SNE (3.96 mg) or SNE (12 mg) adjuvant. See Example 7.

FIG. 13B: graphical representation of HPV VLP 18 antibody levels in Rhesus Macaques after two-doses of a 9vHPV Vaccine combined with either a CLA-SNE (3.96 mg) or SNE (12 mg) adjuvant. See Example 7.

FIG. 14: graphical representation of individual HPV VLP antibody levels in Rhesus Macaques measured at 32 Weeks after two-doses of a 9vHPV Vaccine combined with either 3.96 mg CLA-SNE or 12 mg SNE adjuvant. See Example 7.

### DETAILED DESCRIPTION

In one aspect, the invention provides a composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 and a squalene nanoemulsion (SNE) adjuvant, wherein the SNE adjuvant comprises sorbitan trioleate (SPAN-85); polysorbate-20 (PS-20) or polysorbate-80 (PS-80); and squalene. In some embodiments, the composition also includes an aluminum adjuvant. In some embodiments, the VLPs are adsorbed on an aluminum adjuvant.

### Squalene Nanoemulsion

Squalene nanoemulsions (“SNE”) of the invention refer to a formulation of emulsifiers and/or solubilizers and/or surfactants and/or lipids. In one embodiment, the disclosure provides, among other things, a composition that comprises four SNE components: (1) a cationic lipid; (2) sorbitan trioleate (SPAN-85); (3) polysorbate-20 (PS-20) or polysorbate-80 (PS-80), and (4) squalene. In one embodiment, the SNE composition comprises the cationic lipid (13Z,16Z)-N,N-dimethyl-3-nonyldocosa-13,16-dien-1-amine (“CLA” or, when the cationic lipid is included in the SNE, “CLA-SNE”).

Cationic lipids and methods of making cationic lipids are well known in the art.

In some embodiments, the cationic lipid includes any cationic lipid mentioned in U.S. Patent Application Publication Nos. US 2008/0085870, US 2008/0057080, US 2009/0263407, US 2009/0285881, US 2010/0055168, US 2010/0055169, US 2010/0063135, US 2010/0076055, US 2010/0099738, US 2010/0104629, US 2013/0017239, and US 2016/0361411, International Patent Application Publication No. WO2011/022460; WO2012/040184, WO2011/076807, WO2010/021865, WO 2009/132131, WO2010/042877, WO2010/146740, WO2010/105209, and in U.S. Pat. Nos. 5,208,036, 5,264,618, 5,279,833, 5,283,185, 6,890,557, and 9,669,097.

In some embodiments, cationic lipids useful in the compositions of the invention have the following structure, illustrated by Formula 1:

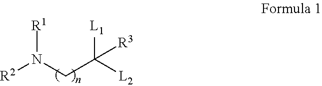

wherein:
R¹ and R² are each methyl;
R³ is H;
n is 1 or 2;
L₁ is selected from C₈-C₂₄ alkyl and C₈-C₂₄ alkenyl; and
L₂ is selected from C₄-C₉ alkyl and C₄-C₉ alkenyl;
or any pharmaceutically acceptable salt or stereoisomer thereof.

In some embodiments, the cationic lipid is an aminoalkyl lipid. In some embodiments, the cationic lipid is an asymmetric aminoalkyl lipid. In an embodiment of the invention, the cationic lipid is (13Z,16Z)-N,N-dimethyl-3-nonyldocosa-13,16-dien-1-amine (CLA); or (6Z,9Z,26Z,29Z)-N,N-dimethylpentatriaconta-6,9,26,29-tetraen-18-amine (CLX); or N,N-dimethyl-1-((1S,2R)-2-octylcyclopropyl) heptadecan-8-amine (CLY).

In another embodiment of the invention, the cationic lipid is selected from: DLinDMA; DLinKC2DMA; DLin-MC3-DMA; CLinDMA; S-Octyl CLinDMA; (2S)-1-{7-[(3P)-cholest-5-en-3-yloxy]heptyloxy}-3-[(4Z)-dec-4-en-1-yloxy]-N,N-dimethylpropan-2-amine; (2R)-1-{4-[(3P)-cholest-5-en-3-yloxy]butoxy}-3-[(4Z)-dec-4-en-1-yloxy]-N,N-dimethylpropan-2-amine; 1-[(2R)-1-{4-[(3β)-cholest-5-en-3-yloxy]butoxy}-3-(octyloxy) propan-2-yl]guanidine; 1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine; 1-[(2R)-1-{4-[(3β)-cholest-5-en-3-yloxy]butoxy}-N,N-dimethyl-3-[(9Z,12Z)-octadeca-9,12-dien-1-yloxy]propan-2-amine; (2S)-1-({6-[(3P))-cholest-5-en-3-yloxy]hexyl}oxy)-N,N-dimethyl-3-[(9Z)-octadec-9-en-1-yloxy]propan-2-amine; (3β)-3-[6-{[(2S)-3-[(9Z)-octadec-9-en-1-yloxyl]-2-(pyrrolidin-1-yl)propyl]oxy}hexyl)oxy]cholest-5-ene; (2R)-1-{4-[(3P)-cholest-5-en-3-yloxy]butoxy}-3-(octyloxy) propan-2-amine; (2R)-1-({8-[(3P)-cholest-5-en-3-yloxy]octyl}oxy)-N,N-dimethyl-3-(pentyloxy) propan-2-amine; (2R)-1-({8-[(3P)-cholest-5-en-3-yloxy]octyl}oxy)-3-(heptyloxy)-N,N-dimethylpropan-2-amine; (2R)-1-({8-[(3P)-cholest-5-en-3-yloxy]octyl}oxy)-N,N-dimethyl-3-[(2Z)-pent-2-en-1-yloxy]propan-2-amine; (2S)-1-butoxy-3-({8-[(3P)-cholest-5-en-3-yloxy]octyl}oxy)-N,N-dimethylpropan-2-amine; (2S-1-({8-[(3P)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine; 2-amino-2-{[(9Z,12Z)-octadeca-9,12-dien-1-yloxy]methyl}propane-1,3-diol; 2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol; 2-amino-3-({6-[(3β,8ξ,9ξ,14ξ,17ξ,20ξ)-cholest-5-en-3-yloxy]nonyl}oxy)-2-{[(9Z)-octadec-9-en-1-yloxy]methyl}propan-1-ol; (20Z,23Z)-N,N-dimethylnonacosa-20,23-dien-10-amine; (17Z,20Z)-N,N-dimethylhexacosa-17,20-dien-9-amine; (16Z,19Z)-N,N-dimethylpentacosa-16,19-dien-8-amine; (13Z,16Z)-N,N-dimethyldocosa-13,16-dien-5-amine; (12Z,15Z)-N,N-dimethylhenicosa-12,15-dien-4-amine; (14Z,17Z)-N,N-dimethyltricosa-14,17-dien-6-amine; (15Z,18Z)-N,N-dimethyltetracosa-15,18-dien-7-amine; (18Z,21Z)-N,N-dimethylheptacosa-18,21-dien-10-amine; (15Z,18Z)-N,N-dimethyltetracosa-15,18-dien-5-amine; (14Z,17Z)-N,N-dimethyltricosa-14,17-dien-4-amine; (19Z,22Z)-N,N-dimethyloctacosa-19,22-dien-9-amine; (18Z,21Z)-N,N-dimethylheptacosa-18,21-dien-8-amine; (17Z,20Z)-N,N-dimethylhexacosa-17,20-dien-7-amine; (16Z,19Z)-N,N-dimethylpentacosa-16,19-dien-6-amine; (22Z,25Z)-N,N-dimethylhentriaconta-22,25-dien-10-amine; (21 Z,24Z)-N,N-dimethyltriaconta-21,24-dien-9-amine; (18Z)-N,N-dimethylheptacos-18-en-10-amine; (17Z)-N,N-dimethylhexacos-17-en-9-amine; (19Z,22Z)-N,N-dimethyloctacosa-19,22-dien-7-amine; N,N-dimethylheptacosan-10-amine; (20Z,23Z)-N-ethyl-N-methylnonacosa-20,23-dien-10-amine; 1-[(11Z,14Z)-1-nonylicosa-11,14-dien-1-yl]pyrrolidine; (20Z)-N,N-dimethylheptacos-20-en-10-amine; (15Z)-N,N-dimethylheptacos-15-en-10-amine; (14Z)-N,N-dimethylnonacos-14-en-10-amine; (17Z)-N,N-dimethylnonacos-17-en-10-amine; (24Z)-N,N-dimethyltritriacont-24-en-10-amine; (20Z)-N,N-dimethylnonacos-20-en-10-amine; (22Z)-N,N-dimethylhentriacont-22-en-10-amine; (16Z)-N,N-dimethylpentacos-16-en-8-amine; (12Z,15Z)-N,N-dimethyl-2-nonylhenicosa-12,15-dien-1-amine; (13Z,16Z)-N,N-dimethyl-3-nonyldocosa-13,16-dien-1-amine; N,N-dimethyl-1-[(1S,2R)-2-octylcyclopropyl]heptadecan-8-amine; 1-[(1 S,2R)-2-hexylcyclopropyl]-N,N-dimethylnonadecan-10-amine; N,N-dimethyl-1-[(1S,2R)-2-octylcyclopropyl]nonadecan-10-amine; N,N-dimethyl-21-[(1S,2R)-2-octylcyclopropyl]henicosan-10-amine; N,N-dimethyl-1-[(1S,2S)-2-{[(1R,2R)-2-pentylcycolporpyl]methyl}cyclopropyl]nonadecan-10-amine; N,N-dimethyl-1-[(1S,2R)-2-octylcyclopropyl]hexadecan-8-amine; N,N-dimethyl-1-[(1R,2S)-2-undecylcyclopropyl]tetradecan-5-amine; N,N-dimethyl-3-{7-[(1 S,2R)-2-octylcyclopropyl]heptyl}dodecan-1-amine; 1-[(1R,2S)-2-heptylcyclopropyl]-N,N-dimethyloctadecan-9-amine; 1-[(1S,2R)-2-decylcyclopropyl]-N,N-dimethylpentadecan-6-amine; N,N-dimethyl-1-[(1S,2R)-2-octylcyclopropyl]pentadecan-8-amine; and (11E,20Z,23Z)-N,N-dimethylnonacosa-11,20,23-trien-10-amine; or a pharmaceutically acceptable salt thereof, or a stereoisomer of any of the foregoing.

In another embodiment of the invention, the cationic lipid is (13Z,16Z)-N,N-dimethyl-3-nonyldocosa-13,16-dien-1-amine, or a pharmaceutically acceptable salt or stereoisomer thereof.

In another embodiment of the invention, the cationic lipid is (13Z,16Z)-N,N-dimethyl-3-nonyldocosa-13,16-dien-1-amine (CLA).

In some embodiments, the disclosure provides, among other things, a composition that comprises three SNE components: (1) sorbitan trioleate (SPAN-85), (2) polysorbate-20 (PS-20) or polysorbate-80 (PS-80), and (3) squalene. In embodiments of this aspect of the invention, the SNE does not comprise a cationic lipid.

In some embodiments, the SNE comprises 32-97 mole % squalene, 1-34 mole % SPAN-85 and 1-34 mole % of PS-20 or PS-80.

In some embodiments, the SNE comprises 86-98 mole % squalene, 1-7 mole % SPAN-85 and 1-7 mole % of PS-20 or PS-80.

In some embodiments, the SNE comprises 92-94 mole % squalene, 3-4 mole % SPAN-85 and 3-4 mole % of PS-20 or PS-80.

In one embodiment of the invention, the SNE comprises 92.91 mole % squalene, 3.98 mole % SPAN-85 and 3.11 mole % of PS-20 or PS-80.

The disclosure also provides, among other things, a composition that comprises four SNE components: (1) a cationic lipid; (2) sorbitan trioleate (SPAN-85); (3) polysorbate-20 (PS-20) or polysorbate-80 (PS-80), and (4) squalene. A particular SNE composition comprises the cationic lipid (13Z,16Z)-N,N-dimethyl-3-nonyldocosa-13,16-dien-1-amine (“CLA” or, when the cationic lipid is included in the SNE, “CLA-SNE”).

In some embodiments, the SNE comprises 1-60 mole % cationic lipid, 32-97 mole % squalene, 1-4 mole % SPAN-85 and 1-4 mole % of PS-20 or PS-80.

In some embodiments, the SNE comprises 10-14 mole % cationic lipid, 78-84 mole % squalene, 1-6 mole % SPAN-85 and 1-6 mole % of PS-20 or PS-80.

In some embodiments, the SNE comprises 40-46 mole % cationic lipid, 44-52 mole % squalene, 1-8 mole % SPAN-85 and 1-8 mole % of PS-20 or PS-80.

In one embodiment of the invention, the SNE comprises 13.82 mole % cationic lipid, 80.07 mole % squalene, 3.43 mole % SPAN-85 and 2.68 mole % of PS-20 or PS-80.

In one embodiment of the invention, the SNE comprises 44.5 mole % cationic lipid, 51.56 mole % squalene, 2.21 mole % SPAN-85 and 1.72 mole % of PS-20 or PS-80.

In some embodiments, the SNE comprises PS-20.

In some embodiments, the SNE comprises PS-80.

In some embodiments, the disclosure provides, among other things, a composition that comprises one or more non-cationic lipids which can be selected from a surfactant, a mixture of surfactants, a phospholipid, a terpene, a terpenoid, a triterpene or a combination thereof.

In some embodiments, the surfactant is selected from the group consisting of: the polyoxyethylene sorbitan esters surfactants (commonly referred to as the Tweens), especially PS-20 and PS-80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy) polyethoxyethanol (IGEPAL CA-630/NP-40); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (SPAN-85, Tween-85 or [2-[(2R,3S,4R)-4-hydroxy-3-[(Z)-octadec-9-enoyl]oxyoxolan-2-yl]-2-[(Z)-octadec-9-enoyl]oxyethyl] (Z)-octadec-9-enoate) and sorbitan monolaurate.

In some embodiments, mixtures of surfactants are used, e.g., PS-20/SPAN 85 or PS-80/SPAN 85 mixtures. A combination of a polyoxyethylene sorbitan ester such as polyoxyethylene sorbitan monooleate (PS-80) and an octoxynol such as t-octylphenoxypolyethoxyethanol (Triton X-100) are also suitable. Another useful combination comprises laureth 9 plus a polyoxyethylene sorbitan ester and/or an octoxynol.

In some embodiments, the amounts of surfactants or emulsifiers are: polyoxyethylene sorbitan esters (such as PS-20 or PS-80) 0.01 to 10 mole %, in particular about 1 to 4 mole %; octyl- or nonylphenoxy polyoxyethanols (such as Triton X-100, or other detergents in the Triton series) 0.001 to 10 mole %, in particular about 1 to 4 mole %; w/v, in particular 0.01 to 0.1% w/v; polyoxyethylene ethers (such as laureth 9) 0.1 to 20 mole %, preferably 0.5 to 10 mole % and in particular 1 to 4% mole % or about 10% by mass.

In some embodiments, the phospholipid is selected from natural phospholipids including phosphatidylcholine (PC), phosphatidylethanolamine (PE), and phosphatidylglycerol (PG), phosphatidylserine (PS), phosphatidylinositol (PI), phosphatidic acid (phosphatidate) (PA), dipalmitoylphosphatidylcholine, monoacyl-phosphatidylcholine (lyso PC), 1-palmitoyl-2-oleoyl-sn-glycero-3-phosphocholine (POPC), N-Acyl-PE, phosphoinositides, and phosphosphingolipids. Phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS). Fatty acids include C14:0, palmitic acid (C16:0), stearic acid (C18:0), oleic acid (C18:1), linoleic acid (C18:2), linolenic acid (C18:3), and arachidonic acid (C20:4), C20:0, C22:0 and lethicin. In certain embodiments of the invention, the phospholipid is phosphatidylserine, 1,2-distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroylphosphatidylcholine (DLPC), 1,2-dieicosenoyl-sn-glycero-3-phosphocholine, or 1,2-diolcoyl-sn-glycero-3-phosphocholine (DOPC).

In some embodiments, the terpine is selected from monoterpenes including geraniol, terpineol, limonene, myrcene, linalool or pinene; sesquiterpenes including humulene, farnesenes, farnesol; diterpenes including cafestol, kahweol, cembrene and taxadiene, triterpenes including squalene and squalane; tetraterpenes including acyclic lycopene, the monocyclic gamma-carotene, and the bicyclic alpha- and beta-carotenes; polyterpenes and norisoprenoids. In some embodiments, the terpine is squalene.

In one embodiment of the invention, the SNE comprises 50-85 mole % squalene, and 1-10 mole % non-ionic surfactants. In one aspect of this embodiment, the non-ionic surfactant comprises a mixture of PS-20 and SPAN-85 or a mixture of PS-80 and SPAN-85.

In one embodiment of the invention, the SNE comprises 0-45 mole % cationic lipid, 50-85 mole % squalene, and 1-10 mole % non-ionic surfactants. In one aspect of this embodiment, the non-ionic surfactant comprises a mixture of PS-20 and SPAN-85 or a mixture of PS-80 and SPAN-85.

In one embodiment of the invention, the SNE comprises one or more cationic lipids, one or more terpenes (e.g., squalene), and/or one or more sorbitan-based surfactants (e.g., PS-20 or PS-80; SPAN-85) at specific molar ratios.

General Methods of Making SNEs (with and without a Cationic Lipid)

Generally, SNEs may be formed, for example, by initially combining and mixing the lipid components together, or initially utilizing a single lipid, such as a cationic lipid. Once mixed and blended (when combining and mixing lipid components together), an aqueous buffer is added and mixed with the initial lipid or lipid components to form a blended emulsion mixture. The blended emulsion components are first subjected to course homogenization followed by fine homogenization. Then, the resulting formulation is subjected to a final filtration step and stored at 4° C. A lipid solution may include one or more cationic lipids, one or more terpenes (e.g., squalene), one or more sorbitan-based surfactants (e.g., PS-20 or PS-80; SPAN-85) at specific molar ratios.

### The VLPs

As stated above, the pharmaceutical compositions and formulations of the invention comprise at least one HPV VLP type, such as HPV 16 or 18. In particular embodiments of the compositions disclosed herein, the vaccine further comprises VLPs of at least one additional HPV type. In further embodiments, the at least one additional HPV type is selected from the group consisting of: 6, 11, 16, 18, 26, 31, 33, 35, 39, 45, 51, 52, 53, 55, 56, 58, 59, 66, 68, 69, 70, 73, and 82. In some embodiments, the at least one HPV type includes HPV 16 and 18. In some embodiments, the at least one HPV type includes HPV 6, 11, 16, and 18. In some embodiments, the at least one HPV type includes HPV 6, 18, 52, and 58. In some embodiments, the at least one HPV type includes HPV 6, 11, 16, 18, 31, 45, 52, and 58. In some embodiments, the at least one HPV type includes HPV 6, 11, 16, 18, 33, 45, 52, and 58. In some embodiments, the at least one HPV type includes HPV 6, 11, 16, 18, 31, 33, 45, 52, and 58. In some embodiments, the at least one HPV type includes 6, 11, 16, 18, 31, 33, 45, 52, and 59. In some embodiments, the at least one HPV type includes HPV 6, 11, 16, 18, 31, 33, 45, 53, and 58. In some embodiments, the at least one HPV type includes HPV 6, 11, 16, 18, 31, 33, 45, 53, and 59. In some embodiments, the at least one HPV type includes HPV 6, 11, 16, 18, 31, 33, 35, 45, 52, and 58. In some embodiments, the at least one HPV type includes HPV 6, 11, 16, 18, 31, 33, 35, 45, 52, 58, and 59. In some embodiments, the at least one HPV type includes HPV 6, 11, 16, 18, 31, 33, 45, 52, 58, 59, and 68. In some embodiments, the at least one HPV type includes HPV 6, 11, 16, 18, 31, 33, 35, 39, 45, 51, 52, 56, 58, and 59. In some embodiments, the at least one HPV type includes HPV 6, 11, 16, 18, 26, 31, 33, 35, 45, 51, 52, 58, 59, and 69. In some embodiments, the at least one HPV type includes HPV 6, 11, 16, 18, 31, 33, 35, 39, 45, 51, 52, 56, 58, 59, and 68. In some embodiments, the at least one HPV type includes HPV 6, 11, 16, 18, 31, 33, 35, 39, 45, 51, 52, 56, 58, 59, and 73. In some embodiments, the at least one HPV type includes HPV 6, 11, 16, 18, 26, 31, 33, 35, 39, 45, 51, 52, 58, 59, 68, 69, and 70. In some embodiments, the at least one HPV type includes HPV 6, 11, 16, 18, 31, 33, 35, 39, 45, 51, 52, 56, 58, 59, 68, and 73. In some embodiments, the at least one HPV type includes HPV 6, 11, 16, 18, 26, 31, 33, 35, 39, 45, 51, 52, 53, 56, 58, 59, 66, 68, 69, and 70. In some embodiments, the at least one HPV type includes HPV 6, 11, 16, 18, 26, 31, 33, 35, 39, 45, 51, 52, 53, 56, 58, 59, 66, 68, 69, 70, and 73.

The pharmaceutical compositions of the invention comprise HPV VLPs comprised of recombinant L1 or recombinant L1+L2 proteins of HPV. HPV L1 or L1+L2 protein can be expressed recombinantly by molecular cloning of L1 or L1+L2 DNA into an expression vector containing a suitable promoter and other appropriate transcription regulatory elements, and transferred into prokaryotic or eukaryotic host cells to produce recombinant protein. Techniques for such manipulations are fully described by Sambrook et al. (Molecular Cloning: A Laboratory Manual; Cold Spring Harbor Laboratory, Cold Spring Harbor, New York, (1989)), which is hereby incorporated by reference. VLPs can self-assemble when L1 protein is recombinantly expressed in a host cell.

The recombinant HPV L1 proteins of the invention may be any full-length L1 protein sequence that can be found in nature or any mutated or truncated L1 protein that is capable of self-assembling into VLPs. In particular embodiments of the invention, the pharmaceutical compositions and vaccines described herein comprise HPV VLPs comprised of recombinant HPV L1 protein and do not contain HPV L2 protein. In certain embodiments, the vaccine compositions or pharmaceutical compositions described herein comprise HPV VLPs comprised of a full-length recombinant HPV L1 protein. In other embodiments, the HPV VLPs are comprised of truncated HPV L1 protein, e.g., L1 protein that are truncated at the C-terminal end. L1 protein sequences for use in the invention can be determined by isolating DNA from one or more clinical samples containing an HPV type of choice, determining the sequence of the HPV L1 DNA sequence, and translating the DNA sequence into an amino acid sequence using the genetic code. Many exemplary L1 sequences suitable for use in the invention can be found in the literature. Sec, e.g., U.S. Pat. Nos. 5,820,870; 7,250,170; 7,276,243; 7,482,428; 7,976,848; 7,498,036; 7,700,103; 7,744,892; and U.S. Pat. No. 5,437,951; Kirii et al. (Virology 185 (1): 424-427 (1991)). Further L1 proteins that are useful in the compositions and formulations of the invention include biologically active fragments and/or mutants of an HPV L1 sequence, including but not necessarily limited to amino acid substitutions, deletions, additions, amino terminal truncations and carboxy-terminal truncations, such that these mutations provide L1 proteins or protein fragments that are capable of forming a VLP. See, e.g., International Publication WO 2006/114312 and U.S. Pat. No. 6,599,508. Appropriate host cells for the expression of recombinant HPV L1 or recombinant L1+L2 and subsequent self-assembly of VLPs include, but are not limited to yeast cells, insect cells, mammalian cells or bacteria. In exemplary embodiments of the invention, the VLPs are produced in yeast cells such as a yeast selected from the group consisting of: Saccharomyces cerevisiae, Hansenula polymorpha, Pichia pastoris, Kluyvermyces fragilis, Kluyveromyces lactis, and Schizosaccharomyces pombe. In particular embodiments, the HPV VLPs are produced in Saccharomyces cerevisiae cells. Expression of HPV VLPs in yeast cells offers the advantages of being cost-effective and easily adapted to large-scale growth in fermenters.

The invention also includes pharmaceutical compositions comprising mutant forms of HPV VLPs, such as HPV VLPs that comprise biologically active fragments and/or mutants of an HPV L1 and/or L2 protein, including but not necessarily limited to amino acid substitutions, deletions, additions, amino terminal truncations and carboxy-terminal truncations such that these mutations provide proteins or protein fragments of therapeutic or prophylactic use and would be useful for HPV VLP vaccine development. Any such mutant form of an HPV L1 protein should be capable of forming VLPs and of provoking an immune response against the desired HPV type when administered to a human.

Additionally, one of skill in the art will recognize that the HPV L1 or L1+L2 proteins, which are used to self-assemble VLPs for inclusion in the compositions disclosed herein, may be encoded by a full-length wild-type HPV L1 or L2 polynucleotide, or may be encoded by a fragment or mutant of the known wild-type sequence. Wild-type polynucleotide sequences that encode mRNA expressing HPV L1 or L2 protein are available in the art. Any mutant polynucleotide will encode either a protein or protein fragment which at least substantially mimics the pharmacological properties of an HPV L1 or L2 protein, including the ability to form VLPs that are able to provoke an immune response against the HPV type of interest when administered to a human. Any such polynucleotide includes but is not necessarily limited to: nucleotide substitutions, deletions, additions, amino-terminal truncations and carboxy-terminal truncations.

The amount of virus-like particles of each HPV type to be included in the formulations and compositions of the invention will depend on the immunogenicity of the expressed gene product. In general, a therapeutically effective dose of VLPs of any of the at least one HPV type is about 1 μg to about 300 μg. In some embodiments, a therapeutically effective dose of VLPs of any of the at least one HPV type is about 1 μg to 200 μg. In some embodiments, a therapeutically effective dose of VLPs of any of the at least one HPV type is about 1 μg to 100 μg. In some embodiments, a therapeutically effective dose of VLPs of any of the at least one HPV type is about 5 μg to 200 μg. In some embodiments, a therapeutically effective dose of VLPs of any of the at least one HPV type is about 5 μg to 100 μg. In some embodiments, a therapeutically effective dose of VLPs of any of the at least one HPV type is about 10 μg to 200 μg. In some embodiments, a therapeutically effective dose of VLPs of any of the at least one HPV type is about 10 μg to 100 μg. In some embodiments, a therapeutically effective dose of VLPs of any of the at least one HPV type is about 10 μg to 80 μg. In some embodiments, a therapeutically effective dose of VLPs of any of the at least one HPV type is about preferably about 20 μg to 60 μg.

In some embodiments, a dose of a composition is in the range of about 0.10 mL to about 1.5 mL. In some embodiments, a dose is the range of about 0.25 mL to about 1.25 mL. In some embodiments, a dose is in the range of about 0.5 mL to about 1.0 mL. In some embodiments, the dose is a 0.25 mL dose. In some embodiments, the dose is a 0.5 mL dose. In some embodiments, the dose is a 0.75 mL dose. In some embodiments, the dose is a 1.0 mL dose. In some embodiments, the dose is a 1.25 mL dose.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein, and
10-120 μg of VLPs of HPV Type 58 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein,
10-120 μg of VLPs of HPV Type 58 L1 protein,
10-120 μg of VLPs of HPV Type 59 L1 protein,
10-120 μg of VLPs of HPV Type 68 L1 protein,
10-120 μg of VLPs of HPV Type 69 L1 protein,
10-120 μg of VLPs of HPV Type 70 L1 protein, and
10-120 μg of VLPs of HPV Type 73 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 μg of VLPs of HPV Type 51 L1 protein,
10-120 μg of VLPs of HPV Type 52 L1 protein,
10-120 μg of VLPs of HPV Type 56 L1 protein,
10-120 μg of VLPs of HPV Type 58 L1 protein, and
10-120 μg of VLPs of HPV Type 59 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein,
10-120 μg of VLPs of HPV Type 56 L1 protein,
10-120 μg of VLPs of HPV Type 58 L1 protein,
10-120 μg of VLPs of HPV Type 59 L1 protein,
10-120 μg of VLPs of HPV Type 68 L1 protein,
10-120 μg of VLPs of HPV Type 70 L1 protein, and
10-120 μg of VLPs of HPV Type 73 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 μg of VLPs of HPV Type 51 L1 protein,
10-120 μg of VLPs of HPV Type 52 L1 protein,
10-120 μg of VLPs of HPV Type 56 L1 protein,
10-120 μg of VLPs of HPV Type 58 L1 protein, and
10-120 μg of VLPs of HPV Type 69 L1 protein.

In some embodiments, a dose of a composition or vaccine including VLPs of the at least one HPV type includes:
15-160 g of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 μg of VLPs of HPV Type 51 L1 protein,
10-120 μg of VLPs of HPV Type 52 L1 protein,
10-120 μg of VLPs of HPV Type 58 L1 protein,
10-120 μg of VLPs of HPV Type 59 L1 protein, and
10-120 μg of VLPs of HPV Type 69 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 μg of VLPs of HPV Type 51 L1 protein,
10-120 μg of VLPs of HPV Type 52 L1 protein,
10-120 g of VLPs of HPV Type 56 L1 protein,
10-120 μg of VLPs of HPV Type 58 L1 protein,
10-120 μg of VLPs of HPV Type 59 L1 protein,
10-120 μg of VLPs of HPV Type 68 L1 protein,
10-120 μg of VLPs of HPV Type 69 L1 protein, and
10-120 μg of VLPs of HPV Type 70 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 g of VLPs of HPV Type 51 L1 protein,
10-120 μg of VLPs of HPV Type 52 L1 protein,
10-120 μg of VLPs of HPV Type 56 L1 protein,
10-120 μg of VLPs of HPV Type 58 L1 protein,
10-120 μg of VLPs of HPV Type 59 L1 protein,
10-120 μg of VLPs of HPV Type 66 L1 protein,
10-120 μg of VLPs of HPV Type 68 L1 protein,
10-120 μg of VLPs of HPV Type 69 L1 protein,
10-120 μg of VLPs of HPV Type 70 L1 protein.

In some embodiments, a dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 35 L1 protein,
10-120 μg of VLPs of HPV Type 39 L1 protein,
10-120 μg of VLPs of HPV Type 45 L1 protein,
10-120 μg of VLPs of HPV Type 51 L1 protein,
10-120 μg of VLPs of HPV Type 52 L1 protein,
10-120 μg of VLPs of HPV Type 56 L1 protein,
10-120 μg of VLPs of HPV Type 58 L1 protein,
10-120 μg of VLPs of HPV Type 59 L1 protein,
10-120 μg of VLPs of HPV Type 66 L1 protein,
10-120 μg of VLPs of HPV Type 68 L1 protein,
10-120 μg of VLPs of HPV Type 69 L1 protein,
10-120 μg of VLPs of HPV Type 70 L1 protein, and
10-120 μg of VLPs of HPV Type 73 L1 protein.

In some embodiments, a dose of a composition or vaccine including VLPs of the at least one HPV type includes:
15-120 μg of VLPs of HPV Type 6 L1 protein,
20-150 μg of VLPs of HPV Type 11 L1 protein,
30-210 μg of VLPs of HPV Type 16 L1 protein,
20-150 μg of VLPs of HPV Type 18 L1 protein,
10-90 μg of VLPs of HPV Type 31 L1 protein,
10-90 μg of VLPs of HPV Type 33 L1 protein,
10-90 μg of VLPs of HPV Type 45 L1 protein,
10-90 μg of VLPs of HPV Type 52 L1 protein, and
10-90 μg of VLPs of HPV Type 58 L1 protein.

In some embodiments, a dose of a composition or vaccine including VLPs of the at least one HPV type includes:
15-80 μg of VLPs of HPV Type 6 L1 protein,
20-100 μg of VLPs of HPV Type 11 L1 protein,
30-140 μg of VLPs of HPV Type 16 L1 protein,
20-100 μg of VLPs of HPV Type 18 L1 protein,
10-60 μg of VLPs of HPV Type 31 L1 protein,
10-60 μg of VLPs of HPV Type 33 L1 protein,
10-60 μg of VLPs of HPV Type 45 L1 protein,
10-60 μg of VLPs of HPV Type 52 L1 protein, and
10-60 μg of VLPs of HPV Type 58 L1 protein.

In some embodiments, a dose of a composition or vaccine including VLPs of the at least one HPV type includes:
15-40 μg of VLPs of HPV Type 6 L1 protein,
20-50 μg of VLPs of HPV Type 11 L1 protein,
30-70 μg of VLPs of HPV Type 16 L1 protein,
20-50 μg of VLPs of HPV Type 18 L1 protein,
10-30 μg of VLPs of HPV Type 31 L1 protein,
10-30 μg of VLPs of HPV Type 33 L1 protein,
10-30 μg of VLPs of HPV Type 45 L1 protein,
10-30 μg of VLPs of HPV Type 52 L1 protein, and
10-30 μg of VLPs of HPV Type 58 L1 protein.

In some embodiments, a dose of a composition or vaccine including VLPs of the at least one HPV type includes:
90 μg of VLPs of HPV Type 6 L1 protein,
120 μg of VLPs of HPV Type 11 L1 protein,
180 μg of VLPs of HPV Type 16 L1 protein,
120 μg of VLPs of HPV Type 18 L1 protein,
60 μg of VLPs of HPV Type 31 L1 protein,
60 μg of VLPs of HPV Type 33 L1 protein,
60 μg of VLPs of HPV Type 45 L1 protein,
60 μg of VLPs of HPV Type 52 L1 protein, and
60 μg of VLPs of HPV Type 58 L1 protein.

In some embodiments, a dose of a composition or vaccine including VLPs of the at least one HPV type includes:
60 μg of VLPs of HPV Type 6 L1 protein,
80 μg of VLPs of HPV Type 11 L1 protein,
120 μg of VLPs of HPV Type 16 L1 protein,
80 μg of VLPs of HPV Type 18 L1 protein,
40 μg of VLPs of HPV Type 31 L1 protein,
40 μg of VLPs of HPV Type 33 L1 protein,
40 μg of VLPs of HPV Type 45 L1 protein,
40 μg of VLPs of HPV Type 52 L1 protein, and
40 μg of VLPs of HPV Type 58 L1 protein.

In some embodiments, a dose of a composition or vaccine including VLPs of the at least one HPV type includes:
30 μg of VLPs of HPV Type 6 L1 protein,
40 μg of VLPs of HPV Type 11 L1 protein,
60 μg of VLPs of HPV Type 16 L1 protein,
40 μg of VLPs of HPV Type 18 L1 protein,
20 μg of VLPs of HPV Type 31 L1 protein,
20 μg of VLPs of HPV Type 33 L1 protein,
20 μg of VLPs of HPV Type 45 L1 protein,
20 μg of VLPs of HPV Type 52 L1 protein, and
20 μg of VLPs of HPV Type 58 L1 protein.

### The Aluminum Adjuvant

The aluminum adjuvant of the invention may be in the form of aluminum hydroxide (Al(OH)₃), aluminum phosphate (AlPO₄), aluminum hydroxyphosphate, amorphous aluminum hydroxyphosphate sulfate (AAHS) or so-called “alum” (KAl(SO₄)-12H₂O) (see Klein et al., Analysis of aluminum hydroxyphosphate vaccine adjuvants by (27)A1 MAS NMR., J Pharm. Sci. 89(3): 311-21 (2000)). In exemplary embodiments of the invention provided herein, the aluminum adjuvant is aluminum hydroxyphosphate or AAHS. The ratio of phosphate to aluminum in the aluminum adjuvant can range from 0 to 1.3. In embodiments of this aspect of the invention, the phosphate to aluminum ratio is within the range of 0.1 to 0.70. In some embodiments, the phosphate to aluminum ratio is within the range of 0.2 to 0.50.

One of skill in the art will be able to determine an optimal dosage of aluminum adjuvant that is both safe and effective at increasing the immune response to the targeted HPV type(s). For a discussion of the safety profile of aluminum, as well as amounts of aluminum included in FDA-licensed vaccines, see Baylor et al., Vaccine 20: S18-S23 (2002). In some embodiments, the aluminum adjuvant is present in the amount of about 100 to 3600 μg/dose (200 to 7200 μg/mL concentration). In some embodiments, the aluminum adjuvant is present in the amount of about 100 to 2700 μg/dose (200 to 5400 μg/mL concentration). In some embodiments, the aluminum adjuvant is present in the amount of about 100 to 1800 μg/dose (200 to 3600 μg/mL concentration). In some embodiments, the aluminum adjuvant is present in the amount of about 100 to 900 μg/dose (200 to 1800 μg/mL concentration). In some embodiments of the formulations and compositions of the invention, there is between 200 and 300 μg aluminum adjuvant per dose of vaccine. In alternative embodiments of the formulations and compositions of the invention, there is between 300 and 500 μg aluminum adjuvant per dose of vaccine. In alternative embodiments of the formulations and compositions of the invention, there is between 400 and 1200 μg aluminum adjuvant per dose of vaccine. In alternative embodiments of the formulations and compositions of the invention, there is between 1200 and 2000 μg aluminum adjuvant per dose of vaccine. In some embodiments of the formulations and compositions of the invention, there is less than 2000 μg aluminum adjuvant per dose of vaccine. In some embodiments of the formulations and compositions of the invention, there is less than 1500 μg aluminum adjuvant per dose of vaccine. In some embodiments of the formulations and compositions of the invention, there is less than 1000 μg aluminum adjuvant per dose of vaccine. In some embodiments of the formulations and compositions of the invention, there is less than 500 μg aluminum adjuvant per dose of vaccine. In some embodiments of the formulations and compositions of the invention, there is less than 400 μg aluminum adjuvant per dose of vaccine. In some embodiments of the formulations and compositions of the invention, there is less than 300 μg aluminum adjuvant per dose of vaccine. In some embodiments of the formulations and compositions of the invention, there is less than 200 μg aluminum adjuvant per dose of vaccine. In some embodiments of the formulations and compositions of the invention, there is less than 100 μg aluminum adjuvant per dose of vaccine.

### The HPV VLP-Based Vaccine

Any HPV VLP-based vaccine is suitable for use in the pharmaceutical compositions and methods of the invention. Known HPV VLP vaccines can be modified to include both an aluminum adjuvant and an SNE adjuvant. Such HPV VLP vaccines can be field mixed with an SNE adjuvant of the invention (e.g., mixed by the clinician just prior to administration to a patient) or can be formulated together with the SNE adjuvant. New vaccines can be developed according to the invention described herein that comprise at least one HPV type, optionally in the form of an HPV VLP adsorbed to an aluminum adjuvant, in combination with an SNE adjuvant. Additionally, new vaccines can be developed according to the invention described herein that comprise at least one HPV type in the form of an HPV VLP adsorbed to an aluminum adjuvant in combination with an SNE adjuvant.

One exemplary HPV vaccine is a bivalent vaccine protective against HPV 16 and 18, which is known commercially as CERVARIX® (GlaxoSmithKline Biologicals, Rixensart, Belgium). Another exemplary HPV VLP vaccine is a non-infectious recombinant, quadrivalent vaccine prepared from highly purified VLPs of the major capsid (L1) protein of HPV types 6, 11, 16, and 18, and may be referred to herein by its proprietary name GARDASIL® (Merck & Co., Inc., Rahway, NJ, USA), see Bryan, J. T. Vaccine 25 (16): 3001-6 (2007); Shi et al. Clinical Pharmacology and Therapeutics 81 (2): 259-64 (2007). Another exemplary HPV VLP vaccine is the nine-valent vaccine marketed for prevention of HPV (that includes the capsid (L1) protein of HPV types 6, 11, 16, 18, 31, 33, 45, 52, and 58), which is referred to herein by its proprietary name GARDASIL®9 (Merck & Co., Inc., Rahway, NJ, USA).

In some embodiments, the vaccine dose includes, in addition to VLPs, an aluminum adjuvant (as amorphous aluminum hydroxyphosphate sulfate), sodium chloride, L-histidine, polysorbate 80, sodium borate, and water. In some embodiments, the HPV vaccine includes 100-3500 μg aluminum adjuvant, 1-50 mg sodium chloride, 0.05-10 mg L-histidine, 1-100 μg polysorbate, 1-100 μg sodium borate, and water. In some embodiments, the HPV vaccine includes about 500 μg aluminum adjuvant, about 9.56 mg sodium chloride, about 0.78 mg L-histidine, about 50 μg polysorbate 80, about 35 μg sodium borate, and water for injection. Known HPV VLP vaccines can be modified to include both an aluminum adjuvant and an SNE adjuvant in accordance with the invention.

In some embodiments of the invention, the pharmaceutical compositions and formulations comprise HPV VLP-based vaccines, or HPV VLPs as described herein, that are monovalent, bivalent, trivalent, quadrivalent, 5-valent, 6-valent, 7-valent, 8-valent, 9-valent, 10-valent, 11-valent, 12-valent, 13-valent or 14-valent. In particular embodiments, the pharmaceutical compositions and formulations are 9-valent. In other embodiments, the pharmaceutical compositions and formulations are 10-valent. In other embodiments, the pharmaceutical compositions and formulations are 12-valent. In particular embodiments, the pharmaceutical compositions and formulations are 14-valent. In some embodiments, the pharmaceutical compositions comprise HPV VLP-based vaccines, or HPV VLPs as described herein, with more than four different types of HPV VLPs. For example, the pharmaceutical compositions and formulations of the invention may include HPV VLP-based vaccines, or HPV VLPs as described herein, that are 8-valent, 9-valent, 10-valent, and so forth. For example, pharmaceutical compositions comprising VLPs of HPV 16 and/or HPV 18, without the inclusion of other HPV VLP types, are included within the scope of the invention. Multi-valent vaccines comprising HPV VLPs that are different than the HPV types included in GARDASIL® or GARDASIL®9 are also contemplated herein.

In some embodiments, VLPs of HPV types 6 and 11 are included. In some embodiments, VLPs of HPV types 16, 31, and 35 are included. In some embodiments, VLPs of HPV types 18, 45, and 59 are included. In some embodiments, VLPs of HPV types 26, 51, and 69 are included. In some embodiments, VLPs of HPV types 33, 52, and 58 are included. In some embodiments, VLPs of HPV types 39, 68, and 70 are included. In some embodiments, VLPs of HPV types 53, 56, and 66 are included.

In some embodiments, VLPs of HPV types 16 and 18 are included. In some embodiments, VLPs of HPV types 6, 11, 16, and 18 are included. In some embodiments, VLPs of HPV types 6, 18, 52, and 58 are included. In some embodiments, VLPs of HPV types 6, 11, 16, 18, 31, 45, 52, and 58 are included. In some embodiments, VLPs of HPV types 6, 11, 16, 18, 33, 45, 52, and 58 are included. In some embodiments, VLPs of HPV types 6, 11, 16, 18, 31, 33, 45, 52, and 58 are included. In some embodiments, VLPs of HPV types 6, 11, 16, 18, 31, 33, 45, 52, and 59 are included. In some embodiments, VLPs of HPV types 6, 11, 16, 18, 31, 33, 45, 53, and 58 are included. In some embodiments, VLPs of HPV types 6, 11, 16, 18, 31, 33, 45, 53, and 59 are included. In some embodiments, VLPs of HPV types 6, 11, 16, 18, 31, 33, 35, 45, 52, and 58 are included. In some embodiments, VLPs of HPV types 6, 11, 16, 18, 31, 33, 35, 45, 52, 58, and 59 are included. In some embodiments, VLPs of HPV types 6, 11, 16, 18, 31, 33, 45, 52, 58, 59, and 68 are included. In some embodiments, VLPs of HPV types 6, 11, 16, 18, 31, 33, 35, 39, 45, 51, 52, 56, 58, and 59 are included. In some embodiments, VLPs of HPV types 6, 11, 16, 18, 26, 31, 33, 35, 45, 51, 52, 58, 59, and 69 are included. In some embodiments, VLPS of HPV types 6, 11, 16, 18, 31, 33, 35, 39, 45, 51, 52, 56, 58, 59, and 68 are included. In some embodiments, VLPs of HPV types 6, 11, 16, 18, 31, 33, 35, 39, 45, 51, 52, 56, 58, 59, and 73 are included. In some embodiments, VLPs of HPV types 6, 11, 16, 18, 26, 31, 33, 35, 39, 45, 51, 52, 58, 59, 68, 69, and 70 are included. In some embodiments, VLPs of HPV types 6, 11, 16, 18, 31, 33, 35, 39, 45, 51, 52, 56, 58, 59, 68, and 73 are included. In some embodiments, VLPs of HPV types 6, 11, 16, 18, 26, 31, 33, 35, 39, 45, 51, 52, 53, 56, 58, 59, 66, 68, 69, and 70 are included. In some embodiments, VLPs of HPV types 6, 11, 16, 18, 26, 31, 33, 35, 39, 45, 51, 52, 53, 56, 58, 59, 66, 68, 69, 70, and 73 are included.

In some embodiments, the pharmaceutical compositions and formulations comprise HPV VLP-based vaccines and/or antigens as listed in Table I below:

TABLE I

| Name | Antigen | Adjuvant | Party |
|---|---|---|---|
| CERVARIX ® | L1 VLP of HPV-16 and HPV- | Aluminum | GlaxoSmithKline |
| (2vHPV vaccine) | 18 | hydroxide and | Biologics (Rixensart, |
| | | MPL | Belgium) |
| GARDASIL ® | L1 VLP of HPV-6, HPV-11, | AHSS | Merck & Co., Inc., |
| (4vHPV vaccine) | HPV-16 and HPV-18 | | Rahway NJ USA |
| GARDASIL ® 9 | L1 VLP of HPV-6, HPV-11, | AHSS | Merck& Co., Inc., |
| (9vHPV vaccine) | HPV-16, HPV-18, HPV-31, | | Rahway NJ USA |
| | HPV-33, HPV-45, HPV-52 | | |
| | and HPV-58 | | |
| CECOLIN ® | L1 VLP of HPV-16 and HPV- | Aluminum | Xiamen Innovax |
| | 18 | hydroxide | |
| GEOCOLIN ® | L1 VLP of HPV-6 and HPV- | Aluminum | Xiamen Innovax |
| | 11 | hydroxide | |
| L1 capsomers | L1 capsomers of HPV-16 | unknown | R. Garcea, University |
| | | | of Colorado- Boulder |
| RG1-VLP | HPV-16 L1-L2 (17-36) VLP | Aluminum | R. Kirnbauer, NCI, |
| | | hydroxide | Pathovax LLC |
| L2-AAV | L2 peptides of HPV-16 and | unknown | 2A Pharma |
| | HPV-31 displayed on AAV | | |
| | VLP | | |
| L2 multimer | Fusion protein of L2 ~11-88 | Alum | Sanofi, Bravo Vax |
| | of HPV-6, HPV-16, HPV-18, | | |
| | HPV-31 and HPV-39 | | |
| L2-thioredoxin | L2 peptide displayed on | unknown | M. Muller, DKFZ |
| | thioredoxin | | |
| AX03 | L2 peptide displayed on | unknown | Agilvax, NIAID |
| | bacteriophage | | |
| L1-E7 VLP | HPV-16 L1-E7 VLP | None | Medigene AG |
| TA-CIN | HPV-16 L2E7E6 fusion | None | Cantab |
| | protein | | Pharmaceuticals, |
| | | | Xenova |
| TA-GW | HPV-6 L2E7 fusion protein | Aluminum | Cantab |
| | | hydroxide or | Pharmaceuticals, GSK |
| | | AS03 | |

Compositions of the invention may be administered subcutaneously, topically, orally, on the mucosa, intravenously, or intramuscularly. The compositions are administered in an amount sufficient to elicit a protective response. Compositions can be administered by various routes, for example, orally, parenterally, subcutaneously, on the mucosa, or intramuscularly. The dose administered may vary depending on the general condition, sex, weight and age of the patient, and the route of administration.

Compositions of the invention, as highlighted in the various embodiments above, may be referred to as immunogenic compositions.

Compositions of the invention, as highlighted in the various embodiments above may also be referred to as vaccines or vaccine compositions.

In an embodiment, a composition is provided, wherein the SNE comprises PS-20, sorbitan trioleate, squalene and (13Z,16Z)-N,N-dimethyl-3-nonyldocosa 13,16-dien-1-amine.

In an embodiment, a composition is provided, wherein the SNE comprises 5-15 mol % sorbitan trioleate, 25-35 mole % PS-20 or PS-80, 1-2.5 mol % squalene, and 55-65 mol % (13Z,16Z)-N,N-dimethyl-3-nonyldocosa 13,16-dien-1-amine.

In an embodiment, a composition is provided, wherein the SNE, including the cationic lipid, comprises up to 75 mol % of cationic lipid, up to 30 mol % of sorbitan trioleate, up to 30 mol % of polysorbate-20 or polysorbate-80 and 25-85 mol % of squalene.

In an embodiment, a composition is provided, wherein the SNE, including the cationic lipid, comprises up to 50 mol % of cationic lipid, up to 10 mol % of sorbitan trioleate, up to 10 mol % of polysorbate-20 or polysorbate-80 and 50-80 mol % of squalene.

In an embodiment, a composition is provided, wherein the SNE, including the cationic lipid, comprises up to 24 mol % of cationic lipid, 1-8 mol % of sorbitan trioleate, 1-8 mol % of polysorbate-20 or polysorbate-80 and 60-75 mol % of squalene.

In an embodiment, a composition is provided, wherein the SNE, including the cationic lipid, comprises about 10-14 mol % of cationic lipid, 1-4 mol % of sorbitan trioleate, 1-4 mol % of polysorbate-20 or polysorbate-80 and 50-80 mol % of squalene.

In an embodiment, a composition is provided, wherein the SNE, including the cationic lipid, comprises 30-65 mol % cationic lipid, 5-30 mol % sorbitan trioleate, 10-40 mol % squalene, and 0.5-4 mol % PS-20 or PS-80.

In an embodiment, a composition is provided, wherein the SNE, including the cationic lipid, comprises 55-65 mol % cationic lipid, 5-15 mol % sorbitan trioleate, 25-35 mol % squalene, and 1-2.5 mol % PS-20 or PS-80.

In an embodiment, a composition is provided, wherein the SNE, including the cationic lipid, comprises 13-45 mol % cationic lipid, 2-4 mol % sorbitan trioleate, 50-82 mol % squalene, and 1.5-3 mol % PS-20 or PS-80.

In an embodiment, a composition is provided, wherein the SNE, including the cationic lipid, comprises 13-14 mol % cationic lipid, 1-2 mol % sorbitan trioleate, 79-81 mol % squalene, and 1-2 mol % PS-20 or PS-80.

In an embodiment, a composition is provided, wherein the SNE, including the cationic lipid, comprises 0 mol % cationic lipid, 8-10 mol % sorbitan trioleate, 80-84 mol % squalene, and 8-10 mol % PS-20 or PS-80.

In an embodiment, a composition is provided, wherein the SNE, including the cationic lipid, comprises 20 mol % cationic lipid, 30 mol % sorbitan trioleate, 20 mol % squalene, and 30 mol % PS-20 or PS-80.

In an embodiment, a composition is provided, wherein the SNE, including the cationic lipid, comprises about 2 mol % cationic lipid, about 8 mol % sorbitan trioleate, about 82 mol % squalene, and about 8 mol % PS-20 or PS-80.

In an embodiment, a composition is provided, wherein the SNE, including the cationic lipid, comprises 2 mol % cationic lipid, 8 mol % sorbitan trioleate, 82 mol % squalene, and 8 mol % PS-20 or PS-80.

In an embodiment, a composition is provided, wherein the SNE, including the cationic lipid, comprises about 13.82 mol % cationic lipid, about 3.43 mol % sorbitan trioleate, about 80.07 mol % squalene, and about 2.68 mol % PS-20 or PS-80.

In an embodiment, a composition is provided, wherein the SNE, including the cationic lipid, comprises 13.82 mol % cationic lipid, 3.43 mol % sorbitan trioleate, 80.07 mol % squalene, and 2.68 mol % PS-20 or PS-80.

In an embodiment, a composition is provided, wherein the SNE, including the cationic lipid, comprises about 44.5 mol % cationic lipid, about 2.21 mol % sorbitan trioleate, about 51.56 mol % squalene, and about 1.72 mol % PS-20 or PS-80.

In an embodiment, a composition is provided, wherein the SNE, including the cationic lipid, comprises 44.5 mol % cationic lipid, 2.21 mol % sorbitan trioleate, 51.56 mol % squalene, and 1.72 mol % PS-20 or PS-80.

In an embodiment, a composition is provided, wherein the SNE comprises 32 mole % squalene, 34 mole % SPAN-85 and 34 mole % of PS-20 or PS-80.

In an embodiment, a composition is provided, wherein the SNE comprises 98 mole % squalene, 1 mole % SPAN-85 and 1 mole % of PS-20 or PS-80.

In an embodiment, a composition is provided, wherein the SNE comprises 86 mole % squalene, 7 mole % SPAN-85 and 7 mole % of PS-20 or PS-80.

In an embodiment, a composition is provided, wherein the SNE comprises 92 mole % squalene, 4 mole % SPAN-85 and 4 mole % of PS-20 or PS-80.

In an embodiment, a composition is provided, wherein the SNE comprises 94 mole % squalene, 3 mole % SPAN-85 and 3 mole % of PS-20 or PS-80.

In an embodiment, a composition is provided, wherein the SNE comprises 92.91 mole % squalene, 3.98 mole % SPAN-85 and 3.11 mole % of PS-20 or PS-80.

In an embodiment, a composition is provided, wherein the SNE comprises 62 mole % squalene, 17 mole % SPAN-85 and 17 mole % of PS-20 or PS-80.

In each of the embodiments described above the composition further comprises HPV VLPs of at least one HPV type.

In some embodiments, a vaccine composition is provided that includes (1) about 2 μg/mL to about 400 mg/mL cationic lipid, and (2) HPV VLPs of at least one HPV type, wherein each of the HPV VLPs, when present in the vaccine composition, are present in a concentration of about 1 μg to about 300 μg per 0.5 mL of the vaccine composition and wherein the total VLP concentration is between about 10 μg to about 2000 μg per 0.5 mL of the vaccine composition. In some embodiments, a vaccine composition is provided that includes (1) about 2 μg/mL to about 400 mg/mL cationic lipid, (2) about 100 μg to about 3500 μg aluminum adjuvant, and (3) HPV VLPs of at least one HPV type, wherein each of the HPV VLPs, when present in the vaccine composition, are present in a concentration of about 1 μg to about 180 μg per 0.5 mL of the vaccine composition and wherein the total VLP concentration is between about 10 μg to about 2000 μg per 0.5 mL of the vaccine composition.

In some embodiments, a vaccine composition is provided that includes about 2 μg/mL to about 400 mg/mL cationic lipid, about 1 μg to about 2000 μg HPV VLPs of at least two HPV types, and about 100 μg to about 2700 μg aluminum adjuvant. In some embodiments, a vaccine composition is provided that includes about 2 μg/mL to about 400 mg/mL cationic lipid, HPV VLPs of at least four HPV types, and about 100 μg to about 3500 μg aluminum adjuvant.

In some embodiments, a vaccine composition is provided that includes about 2 μg/mL to about 400 mg/mL cationic lipid, and 1 μg to about 100 μg of each HPV VLP present in the vaccine composition. In some embodiments, a vaccine composition is provided that includes about 2 μg/mL to about 400 mg/mL cationic lipid and 2 μg to about 600 μg of HPV VLPs of two HPV types (i.e., the vaccine is a bivalent HPV VLP vaccine). In some embodiments, a vaccine composition is provided that includes about 2 μg/mL to about 400 mg/mL cationic lipid and 4 μg to about 1200 μg of HPV VLPs of four HPV types (i.e., the vaccine is a quadrivalent HPV VLP vaccine). In some embodiments, a vaccine composition is provided that includes about 2 μg/mL to about 400 mg/mL cationic lipid and 9 μg to about 2700 μg of HPV VLPs of nine HPV types (i.e., the vaccine is 9-valent HPV VLP vaccine). In some embodiments, a vaccine composition is provided that includes about 2 μg/mL to about 400 mg/mL cationic lipid and 20 μg to about 6000 μg of HPV VLPs of twenty HPV types (i.e., the vaccine is a 20-valent HPV VLP vaccine). In some embodiments, the vaccine composition also includes about 100 μg to about 2700 μg aluminum adjuvant.

In some embodiments, a vaccine composition is provided that includes about 2 μg/mL to about 400 mg/mL cationic lipid, 1 μg to about 300 μg of a monovalent HPV VLP, and 100 μg to about 2700 μg aluminum adjuvant. In some embodiments, a vaccine composition is provided that includes about 2 μg/mL to about 400 mg/mL cationic lipid, 1 μg to about 300 μg, per VLP, of a bivalent HPV VLP (i.e., HPV VLPs of two HPV types), and 100 μg to about 3500 μg aluminum adjuvant. In some embodiments, a vaccine composition is provided that includes (1) about 2 μg/mL to about 400 mg/mL cationic lipid, (2) 1 μg to about 300 μg, per VLP, of a quadrivalent HPV VLP (i.e., HPV VLPs of four HPV types), and (3) 100 μg to about 3500 μg aluminum adjuvant. In some embodiments, a vaccine composition is provided that includes (1) about 2 μg/mL to about 400 mg/mL cationic lipid, (2) 1 μg to about 300 μg, per VLP, of a 9-valent HPV VLP (i.e., HPV VLPs of 9 HPV types), and (3) 100 μg to about 3500 μg aluminum adjuvant. In some embodiments, a vaccine composition is provided that includes (1) about 2 μg/mL to about 400 mg/mL cationic lipid, (2) 1 μg to about 300 μg, per VLP, of a 20-valent HPV VLP (i.e., HPV VLPs of 20 HPV types), and (3) 100 μg to about 3500 μg aluminum adjuvant.

In some embodiments, the e vaccine composition includes (1) 1 μg to about 300 μg, per VLP, of HPV VLPs (HPV types 16 and 18) and (2) about 2 μg/mL to about 400 mg/mL cationic lipid. In some embodiments, the vaccine composition includes (1) 1 μg to about 300 μg, per VLP, of HPV VLPs (HPV types 6, 11, 16, and 18) and (2) about 2 μg/mL to about 400 mg/mL cationic lipid. In some embodiments, the vaccine composition includes (1) 1 μg to about 300 μg, per VLP, of HPV VLPs (HPV types 6, 11, 16, 18, 31, 33, 45, 52, and 58) and (2) about 2 μg/mL to about 400 mg/mL cationic lipid.

In some embodiments, the vaccine composition includes 1 μg to about 300 μg, per VLP, of HPV VLPs (HPV types 16 and 18), 100 μg to about 3500 μg of an aluminum adjuvant, and about 2 μg/mL to about 400 mg/mL cationic lipid. In some embodiments, the vaccine composition includes 1 μg to about 300 μg, per VLP, of HPV VLPs (HPV types 6, 11, 16, and 18), 100 μg to about 3500 μg of an aluminum adjuvant, and about 2 μg/mL to about 400 mg/mL cationic lipid. In some embodiments, the vaccine composition includes 1 μg to about 300 μg, per VLP, of HPV VLPs. (HPV types 6, 11, 16, 18, 31, 33, 45, 52, and 58), 100 μg to about 3500 μg of an aluminum adjuvant, and about 2 μg/mL to about 400 mg/mL cationic lipid.

The vaccines of the invention comprise VLPs containing the antigenic determinants required to induce the generation of neutralizing antibodies in the subject. The vaccines are expected to be sufficiently safe to be administered without the risk of clinical infection, have no toxic side effects, are stable, compatible with conventional carriers and can be administered effectively. In some embodiments, an SNE adjuvant of the invention is combined with a Human Papillomavirus Bivalent (Types 16 and 18) Vaccine, Recombinant. In some embodiments, the SNE adjuvant of the invention is combined with CERVARIX®. In some embodiments, an SNE adjuvant of the invention is combined with a Human Papillomavirus Quadrivalent (Types 6, 11, 16, 18) Vaccine, Recombinant. In some embodiments, an SNE adjuvant of the invention is combined with GARDASIL®. In some embodiments, an SNE adjuvant of the invention is combined with a Human Papillomavirus 9-valent Vaccine, Recombinant. In some embodiments, an SNE adjuvant of the invention is combined with GARDASIL® 9.

Pharmaceutical compositions, formulations, and vaccines of the invention may be administered subcutaneously, topically, orally, on the mucosa, intravenously, or intramuscularly. The pharmaceutical compositions, formulations, and vaccines are administered in an amount sufficient to elicit a protective response. Vaccines, pharmaceutical compositions and formulations can be administered by various routes, for example, orally, parenterally, subcutaneously, on the mucosa, or intramuscularly. The dose administered may vary depending on the general condition, sex, weight and age of the patient, the route of administration and the type of HPV VLP in the vaccine. The vaccine, pharmaceutical composition, or formulation may be in the form of a capsule, suspension, elixir or solution. Such vaccine, pharmaceutical compositions or formulations may be formulated with an immunologically acceptable carrier.

### Kits of the Invention

Also provided herein are kits including any of the pharmaceutical compositions as described above and instructions for use.

Also provided herein are kits including (1) a pharmaceutical composition comprising HPV VLPs of at least one type of HPV and (2) an SNE adjuvant.

In some embodiments of the kits, the pharmaceutical composition of comprises HPV VLPs of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiments, the pharmaceutical composition is an HPV vaccine. In some embodiments, the HPV vaccine is a Human Papillomavirus Bivalent (Types 16 and 18) Vaccine, Recombinant. In some embodiments, the HPV vaccine is CERVARIX®. In some embodiments, the HPV vaccine is a Human Papillomavirus Quadrivalent (Types 6, 11, 16, 18) Vaccine, Recombinant. In some embodiments, the HPV vaccine is GARDASIL®. In some embodiments, the HPV vaccine is a Papillomavirus 9-valent Vaccine, Recombinant. In some embodiments, the HPV vaccine is GARDASIL® 9.

In some embodiments of the kits, the SNE adjuvant is any of the SNE adjuvants described herein above. In some embodiments, the kit includes 0.1 μg to 100 mg of a SNE. In some embodiments, the kit includes 2 μg/mL to about 400 mg/mL cationic lipid. In some embodiments, the kit includes about 0.1 μg/mL to about 400 mg/mL cationic lipid, and further includes SPAN-85, PS-20 or PS-80 and squalene. In another embodiment, the cationic lipid is CLA. In another embodiment, the cationic lipid is CLX. In another embodiment, the cationic lipid is CLY.

In some embodiments, the kit includes about 60 μg/mL to about 2.4 mg/mL cationic lipid, and further includes 6 μg/mL-240 μg/mL SPAN-85, 6 μg/mL-240 μg/mL PS-20 or PS-80 and 60 μg/mL-2.4 mg/mL of squalene. In another embodiment, the cationic lipid is CLA. In another embodiment, the cationic lipid is CLX. In another embodiment, the cationic lipid is CLY. In some embodiments, the kit includes about 6 μg/mL-24 mg/mL SPAN-85, 6 μg/mL-24 mg/mL PS-20 or PS-80 and 60 μg/mL-240 mg/mL of squalene. In some embodiments, the kit includes about 2 μg/mL-24 mg/mL SPAN-85, 2 μg/mL-2.4 mg/mL PS-20 or PS-80 and 20 μg/mL-24 mg/mL of squalene. In another embodiment, the kit includes 6 μg/mL-2.4 mg/mL SPAN-85, 6 μg/mL-2.4 mg/mL PS-20 or PS-80 and 60 μg/mL-24 mg/mL of squalene.

In some embodiments, the kit includes 30 μg/mL to about 2.4 mg/mL cationic lipid, and further includes 6 μg/mL-14 mg/mL SPAN-85, 6 μg/mL-14 mg/mL PS-20 or PS-80 and 60 μg/mL-34 mg/mL of squalene. In another embodiment, the cationic lipid is CLA. In another embodiment, the cationic lipid is CLX. In another embodiment, the cationic lipid is CLY.

In an embodiment, the kit includes an SNE adjuvant, wherein the SNE comprises PS-20, sorbitan trioleate, squalene and (13Z,16Z)-N,N-dimethyl-3-nonyldocosa 13,16-dien-1-amine.

In an embodiment, the kit includes an SNE adjuvant, wherein the SNE comprises 5-15 mol % sorbitan trioleate, 25-35 mole % PS-20 or PS-80, 1-2.5 mol % squalene, and 55-65 mol % (13Z,16Z)-N,N-dimethyl-3-nonyldocosa 13,16-dien-1-amine.

In an embodiment, the kit includes an SNE adjuvant, wherein the SNE, including the cationic lipid, comprises up to 75 mol % of cationic lipid, up to 30 mol % of sorbitan trioleate, up to 30 mol % of polysorbate-20 or polysorbate-80 and 25-85 mol % amount squalene.

In an embodiment, the kit includes an SNE adjuvant, wherein the SNE, including the cationic lipid, comprises up to 50 mol % of cationic lipid, up to 10 mol % of sorbitan trioleate, up to 10 mol % of polysorbate-20 or polysorbate-80 and 50-80 mol % amount squalene.

In an embodiment, the kit includes an SNE adjuvant, wherein the SNE, including the cationic lipid, comprises up to 24 mol % of cationic lipid, 1-8 mol % of sorbitan trioleate, 1-8 mol % of polysorbate-20 or polysorbate-80 and 60-75 mol % amount squalene.

In an embodiment, the kit includes an SNE adjuvant, wherein the SNE, including the cationic lipid, comprises about 10-14 mol % of cationic lipid, 1-4 mol % of sorbitan trioleate, 1-4 mol % of polysorbate-20 or polysorbate-80 and 50-80 mol % amount squalene.

In an embodiment, the kit includes an SNE adjuvant, wherein the SNE, including the cationic lipid, comprises 30-65 mol % cationic lipid, 5-30 mol % sorbitan trioleate, 10-40 mol % squalene, and 0.5-4 mol % PS-20 or PS-80.

In an embodiment, the kit includes an SNE adjuvant, wherein the SNE, including the cationic lipid, comprises 55-65 mol % cationic lipid, 5-15 mol % sorbitan trioleate, 25-35 mol % squalene, and 1-2.5 mol % PS-20 or PS-80.

In an embodiment, the kit includes an SNE adjuvant, wherein the SNE, including the cationic lipid, comprises 13-45 mol % cationic lipid, 2-4 mol % sorbitan trioleate, 50-82 mol % squalene, and 1.5-3 mol % PS-20 or PS-80.

In an embodiment, the kit includes an SNE adjuvant, wherein the SNE, including the cationic lipid, comprises 13-14 mol % cationic lipid, 1-2 mol % sorbitan trioleate, 79-81 mol % squalene, and 1-2 mol % PS-20 or PS-80.

In an embodiment, the kit includes an SNE adjuvant, wherein the SNE, including the cationic lipid, comprises 0 mol % cationic lipid, 8-10 mol % sorbitan trioleate, 80-84 mol % squalene, and 8-10 mol % PS-20 or PS-80.

In an embodiment, the kit includes an SNE adjuvant, wherein the SNE, including the cationic lipid, comprises 20 mol % cationic lipid, 30 mol % sorbitan trioleate, 20 mol % squalene, and 30 mol % PS-20 or PS-80.

In an embodiment, the kit includes an SNE adjuvant, wherein the SNE, including the cationic lipid, comprises about 2 mol % cationic lipid, about 8 mol % sorbitan trioleate, about 82 mol % squalene, and about 8 mol % PS-20 or PS-80.

In an embodiment, the kit includes an SNE adjuvant, wherein the SNE, including the cationic lipid, comprises 2 mol % cationic lipid, 8 mol % sorbitan trioleate, 82 mol % squalene, and 8 mol % PS-20 or PS-80.

In an embodiment, the kit includes an SNE adjuvant, wherein the SNE, including the cationic lipid, comprises about 13.82 mol % cationic lipid, about 3.43 mol % sorbitan trioleate, about 80.07 mol % squalene, and about 2.68 mol % PS-20 or PS-80.

In an embodiment, the kit includes an SNE adjuvant, wherein the SNE, including the cationic lipid, comprises 13.82 mol % cationic lipid, 3.43 mol % sorbitan trioleate, 80.07 mol % squalene, and 2.68 mol % PS-20 or PS-80.

In an embodiment, the kit includes an SNE adjuvant, wherein the SNE, including the cationic lipid, comprises about 44.5 mol % cationic lipid, about 2.21 mol % sorbitan trioleate, about 51.56 mol % squalene, and about 1.72 mol % PS-20 or PS-80.

In an embodiment, the kit includes an SNE adjuvant, wherein the SNE, including the cationic lipid, comprises 44.5 mol % cationic lipid, 2.21 mol % sorbitan trioleate, 51.56 mol % squalene, and 1.72 mol % PS-20 or PS-80.

In an embodiment, the kit includes an SNE adjuvant, wherein the SNE, comprises 32 mole % squalene, 34 mole % SPAN-85 and 34 mole % of PS-20 or PS-80.

In an embodiment, the kit includes an SNE adjuvant, wherein the SNE, comprises 98 mole % squalene, 1 mole % SPAN-85 and 1 mole % of PS-20 or PS-80.

In an embodiment, the kit includes an SNE adjuvant, wherein the SNE, comprises 86 mole % squalene, 7 mole % SPAN-85 and 7 mole % of PS-20 or PS-80.

In an embodiment, the kit includes an SNE adjuvant, wherein the SNE, comprises 92 mole % squalene, 4 mole % SPAN-85 and 4 mole % of PS-20 or PS-80.

In an embodiment, the kit includes an SNE adjuvant, wherein the SNE, comprises 94 mole % squalene, 3 mole % SPAN-85 and 3 mole % of PS-20 or PS-80.

In an embodiment, the kit includes an SNE adjuvant, wherein the SNE, comprises 92.91 mole % squalene, 3.98 mole % SPAN-85 and 3.11 mole % of PS-20 or PS-80.

In an embodiment, the kit includes an SNE adjuvant, wherein the SNE, comprises 62 mole % squalene, 17 mole % SPAN-85 and 17 mole % of PS-20 or PS-80.

In some embodiments, the kit includes a buffer. In some embodiments, the kit includes a tonicity modifier. In some embodiments, the kit includes a detergent.

In some embodiments of the kits, the kit includes a label or packaging insert that includes a description of the components and/or instructions for use in vivo of the components therein. In some embodiments, the kits include instructions for co-administering (or vaccinating) (1) the pharmaceutical composition or HPV Vaccine and (2) the SNE adjuvant. In some embodiments, the kits include instructions for admixing (1) the pharmaceutical composition or HPV vaccine and (2) the SNE adjuvant and subsequentially administering (or vaccinating) the admixture to a patient.

### Methods of Treatment of the Invention

Also provided herein is a method of inducing an immune response to a human papillomavirus (HPV) in a human patient comprising administering to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of inducing an immune response to a human papillomavirus (HPV) in a human patient including administering an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiments, the SNE adjuvant is formulated separately from the VLPs. In some embodiments, the SNE adjuvant is formulated with the VLPs. In some embodiments, the SNE adjuvant and VLPs are field-mixed to form a pharmaceutical composition prior to administration to the patient. In some embodiments, the SNE adjuvant and VLPs are administered sequentially to a patient.

Also provided herein is a method of inducing an immune response to a human papillomavirus (HPV) in a human patient including co-administering to the patient (1) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (2) an SNE adjuvant.

Also provided herein is a method of preventing infection of or reducing the likelihood of infection of a human patient by a human papillomavirus (HPV) including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of delivering a pharmaceutical composition to a subject that induces a neutralizing titer against an HPV antigen in the subject that includes administering to the subject a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the subject, and wherein the pharmaceutical composition provides enhanced or comparable neutralizing titers when compared to the same pharmaceutical composition when the same composition is formulated without an SNE adjuvant.

Also provided herein is a method for preventing cancer of a human patient cancers caused by human papillomavirus (HPV) Types 16, 18, 31, 33, 45, 52, and 58, including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, anal, oropharyngeal, and other head and neck cancers.

Also provided herein is a method for preventing cancer of a human patient caused by HPV Types 6, 11, 16, 18, 31, 33, 45, 52, and 58 including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, and anal precancerous or dysplastic lesions.

Also provided herein is a method for preventing cancer of a human patient caused by HPV Types 6 and 11, including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is genital warts or condyloma acuminata.

Also provided herein is a method for preventing precancerous or dysplastic lesions of a human patient caused by HPV Types 6, 11, 16, 18, 26, 31, 33, 35, 39, 45, 51, 52, 53, 55, 56, 58, 59, 66, 68, 73, and 82, including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the lesions are selected from Cervical intraepithelial neoplasia (CIN) grade 2/3, cervical adenocarcinoma in situ (AIS), Cervical intraepithelial neoplasia (CIN) grade 1, Vulvar intraepithelial neoplasia (VIN) grade 2 and grade 3, Vaginal intraepithelial neoplasia (VaIN) grade 2 and grade 3, Anal intraepithelial neoplasia (AlN) grades 1, 2, and 3. (1.1).

Also provided herein is a method for preventing HPV-related anogenital disease of a human patient caused by HPV Types selected from the group consisting of HPV types: 6, 11, 16, 18, 26, 31, 33, 35, 39, 45, 51, 52, 53, 55, 56, 58, 59, 66, 68, 73, and 82 including administration to the patient a pharmaceutical composition including an SNE adjuvant and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Embodiments of the invention also include one or more of the pharmaceutical compositions described herein (1) for use in, (2) for use as a medicament or composition for, or (3) for use in the preparation of a medicament for: (a) therapy (e.g., of the human body); (b) medicine; (c) induction of an immune response against HPV types included in the vaccine (d) decreasing the likelihood of HPV infection in a patient; (e) prevention of infection with HPV types in the vaccine, (f) prevention or reduction of the likelihood of cervical cancer, (g) prevention or reduction of the likelihood of vulvar cancer, (h) prevention or reduction of the likelihood of vaginal cancer, (i) prevention or reduction of the likelihood of anal cancer, (j) prevention or reduction of the likelihood of oropharyngeal cancer, (k) prevention or reduction of the likelihood of other head and neck cancers, (k) prevention or reduction of the likelihood of precancerous or dysplastic anal lesions, (1) prevention or reduction of the likelihood of genital warts or condyloma acuminata, (m) prevention or reduction of the likelihood of Cervical intraepithelial neoplasia (CIN) grade 2/3 lesions, (n) prevention or reduction of the likelihood of cervical adenocarcinoma in situ (AIS) lesions, (o) prevention or reduction of the likelihood of Cervical intraepithelial neoplasia (CIN) grade 1 lesions, (p) prevention or reduction of the likelihood of Vulvar intraepithelial neoplasia (VIN) grade 2 and grade 3 lesions, (q) prevention or reduction of the likelihood of Vaginal intraepithelial neoplasia (VaIN) grade 2 and grade 3 lesions, (r) prevention or reduction of the likelihood of Anal intraepithelial neoplasia (AlN) grades 1, 2, and 3 lesions.

In embodiment 1, a composition is provided that includes virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 and a squalene nanoemulsion (SNE) adjuvant, wherein the SNE adjuvant comprises sorbitan trioleate (SPAN-85); polysorbate-20 (PS-20) or polysorbate-80 (PS-80); and squalene.

In embodiment 2, the composition of embodiment 1 is provided, wherein the composition is made by mixing an HPV vaccine and a squalene nanoemulsion (SNE) adjuvant; wherein the HPV vaccine comprises HPV VLPs and a pharmaceutically acceptable carrier and the squalene nanoemulsion (SNE) adjuvant comprises sorbitan trioleate (SPAN-85), polysorbate-20 (PS-20) or polysorbate-80 (PS-80), and squalene.

In embodiment 3, the composition of embodiment 1 or 2 is provided, wherein the SNE adjuvant comprises 6 μg/mL-14 mg/mL SPAN-85, 6 μg/mL-14 mg/mL PS-20 or PS-80, and 20 μg/mL-240 mg/mL of squalene.

In embodiment 4, the composition of embodiment 1-3 is provided, wherein the SNE adjuvant further comprises a cationic lipid.

In embodiment 5, the composition of embodiment 4 is provided, wherein the cationic lipid is (13Z,16Z)-N,N-dimethyl-3-nonyldocosa-13,16-dien-1-amine.

In embodiment 6, the composition of embodiments 4 or 5 is provided, wherein the composition comprises 30 μg/mL-4.8 mg/mL of cationic lipid.

In embodiment 7, the composition of any of embodiments 1-6 is provided, wherein the SNE comprises PS-20.

In embodiment 8, the composition of any of embodiments 1-7 is provided, wherein the composition further comprises a buffer.

In embodiment 9, the composition of embodiment 8 is provided, wherein the buffer is selected from the group consisting of: acetic acid, histidine, citrate, Bis-Tris, HEPES, phosphate, MES, sodium chloride, succinate, Tris, and combinations thereof.

In embodiment 10, the composition of embodiment 8 or 9 is provided, wherein the buffer is present in the amount of about 1 mMol to about 100 mMol.

In embodiment 11, the composition of embodiments 1-10 is provided, wherein the composition further comprises a salt.

In embodiment 12, the composition of embodiment 11 is provided, wherein the salt is NaCl.

In embodiment 13, the composition of embodiments 1-12 is provided, wherein the composition further comprises 5 mM-40 mM histidine at pH 5.1-7.0 and 25 mM-300 mM NaCl.

In embodiment 14, the composition of embodiments 1-12 is provided, wherein the composition further comprises about 20 mM histidine at about pH 5.8 and about 75 mM NaCl.

In embodiment 15, the composition of embodiments 1-14 is provided, wherein the composition comprises VLPs of HPV types 16 and 18.

In embodiment 16, the composition of embodiments 1-15 is provided, wherein the composition comprises VLPs of HPV types 6, 11, 16, and 18.

In embodiment 17, the composition of embodiments 1-16 is provided, wherein the composition comprises VLPs of HPV types 31, 45, 52, and 58.

In embodiment 18, the composition of embodiments 1-17 is provided, wherein the composition comprises VLPs of HPV types 6, 11, 16, 18, 31, 33, 45, 52, and 58.

In embodiment 19, the composition of embodiments 1-18 is provided, wherein the composition further comprises aluminum.

In embodiment 20, the composition of embodiments 1-19 is provided, wherein the HPV VLPs comprise recombinant HPV L1 or recombinant HPV L1+L2 protein.

In embodiment 21, the composition of embodiments 1-19 is provided, wherein the HPV VLPs comprise HPV L1 protein and do not comprise HPV L2 protein.

In embodiment 22, the composition of embodiments 1-19 is provided, wherein the HPV VLPs consist of HPV L1 protein.

In embodiment 23, a method of inducing an immune response to a human papillomavirus (HPV) in a human patient is provided comprising administering to the patient the pharmaceutical composition of any of embodiments 1-22.

In embodiment 24, a method of preventing infection of or reducing the likelihood of infection of a human patient by a human papillomavirus (HPV) is provided comprising administration to the patient the pharmaceutical composition of any of embodiments 1-22.

In embodiment 25, a use of the composition of any of embodiments 1-22 is provided for preventing infection of or reducing the likelihood of infection of a human patient by a human papillomavirus (HPV).

In embodiment 26, a kit is provided comprising: (a) a human papillomavirus (HPV) vaccine; and (b) a squalene nanoemulsion (SNE) adjuvant, wherein the SNE adjuvant comprises sorbitan trioleate (SPAN-85); polysorbate-20 (PS-20) or polysorbate-80 (PS-80); and squalene.

In embodiment 27, the kit of embodiment 26 is provided further comprising instructions for administering to a human patient the HPV vaccine and the SNE adjuvant.

All publications mentioned herein are incorporated by-reference for the purpose of describing and disclosing methodologies and materials that might be used in connection with the invention. Nothing herein is to be construed as an admission that the invention is not entitled to antedate such disclosure by virtue of prior invention.

Having described embodiments of the invention with reference to the accompanying drawings, it is to be understood that the invention is not limited to those precise embodiments, and that various changes and modifications may be used by one skilled in the art without departing from the scope or spirit of the invention as defined in the appended claims.

The following examples illustrate, but do not limit the invention.

### EXAMPLES

### Example 1: Preparation of a Squalene Nanoemulsion (SNE) Adjuvant System with and without the Cationic Lipid, (13Z,16Z)-N,N-dimethyl-3-nonyldocosa-13,16-dien-1-amine)

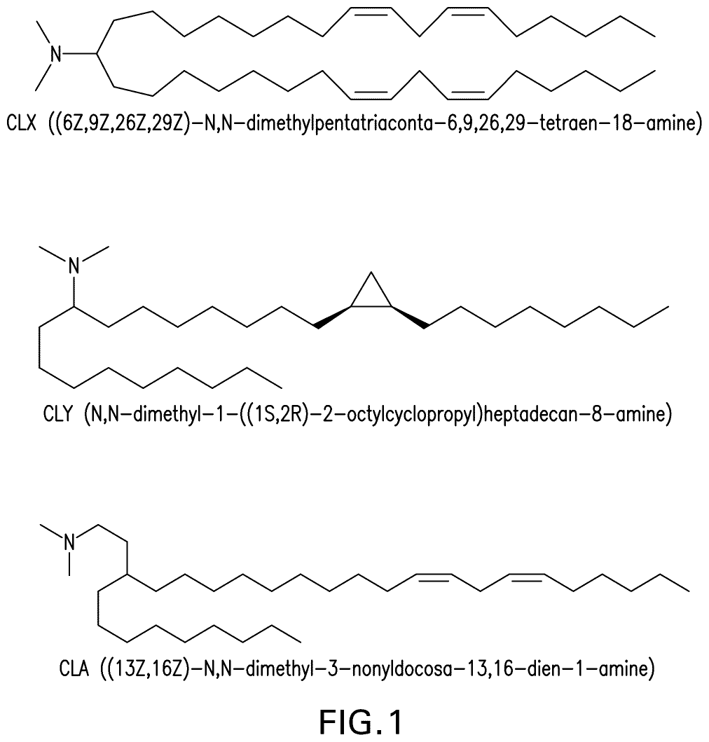

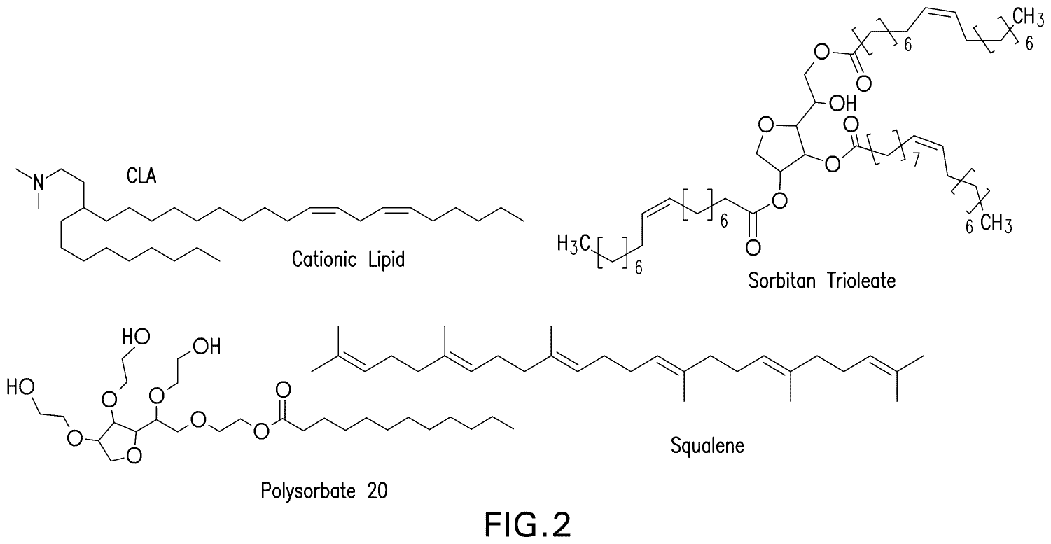

The SNE adjuvant can be prepared with and without cationic lipids, (13Z,16Z)-N,N-dimethyl-3-nonyldocosa-13,16-dien-1-amine) also referred to as CLA, or (6Z,9Z,26Z,29Z)-N,N-dimethylpentatriaconta-6,9,26,29-tetraen-18-amine, also referred to as CLX; or N,N-dimethyl-1-((1S,2R)-2-octylcyclopropyl) heptadecan-8-amine, also referred to as CLY (FIG. 1). The SNE is a multi-component emulsion formulation consisting of 3 stabilizing ingredients; squalene, sorbitan trioleate (SPAN-85), and polysorbate-20 (PS-20) with a cationic lipid, for example, CLA (referred to as CLA-SNE, see Table II) and without a cationic lipid (referred to as SNE, see Table III). This formulation was prepared by combining and mixing the cationic lipid (if used), squalene, SPAN-85 and PS-20 or similar (e.g., surfactants, oils, and solubilizers) components together (Table II and FIG. 2). Once mixed and blended, a histidine buffer was added and mixed with the initial emulsion components. Blended emulsion components were first subjected to course homogenization followed by fine homogenization, as described below. The resulting formulation was subjected to a final 0.2 mm filtration step. Several process parameters within each step, such as order of addition, mixing time, pH, temperature, concentration of components, homogenization, microfluidization were controlled to yield an emulsion system with desired attributes.

TABLE II
Composition of a Representative CLA-SNE Adjuvant

| | | Content of | Molecular | Content of |
|---|---|---|---|---|
| | | Each Lipid | Weight | Each Lipid |
| Component | Description | (Mole %) | (g/mol) | (Mass %) |
| CLA | (13Z,16Z)-N,N- | 13.82-44.5 | 475.9 | 14.29-45.45 |
| | dimethyl- | | | |
| | 3-nonyldocosa- | | | |
| | 13,16-dien-1-amine | | | |
| Squalene | squalene | 51.56-80.07 | 410.72 | 45.45-71.43 |
| SPAN-85 | sorbitan trioleate | 2.21-3.43 | 957.5 | 4.55-7.14 |
| PS-20 | polysorbate-20 | 1.72-2.68 | 1228 | 4.55-7.14 |

| | | |
|---|---|---|
| Buffer | 20 mM Histidine, | N/A |
| Matrix | pH 5.8 | |

TABLE III
Composition of a Representative SNE Adjuvant

| | | Content of | Molecular | Content of |
|---|---|---|---|---|
| | | Each Lipid | Weight | Each Lipid |
| Component | Description | (Mole %) | (g/mol) | (Mass %) |
| Squalene | squalene | 92.25-93.57 | 410.72 | 81.97-84.75 |
| SPAN-85 | sorbitan trioleate | 3.61-4.35 | 957.5 | 7.63-9.02 |
| PS-20 | polysorbate-20 | 2.82-3.39 | 1228 | 7.63-9.02 |

| | | |
|---|---|---|
| Buffer | 20 mM Histidine, | N/A |
| Matrix | pH 5.8 | |

### Formulation Preparation

The squalene and solubilizer formulation (referred to as the oil phase) of the emulsion was prepared by addition of squalene, SPAN-85, PS-20 and CLA to a vessel. The oil phase was then mixed using magnetic stirring at 100-1000 RPM for 10 to 120 minutes. After mixing of these components, an aqueous phase comprised of 20 mM Histidine pH 5.8, was slowly added to the oil phase while being mixed using a magnetic stir bar. This formulation was then mixed again for 1 hour.

### Coarse Homogenization

The oil and aqueous phase mixture (referred to as the pre-homogenized emulsion or PHE) was then homogenized and size reduced to form a rough emulsion using a rotor stator homogenizer at ambient temperature. The homogenizer arm tip was submerged into the PHE and held in place near the bottom of the formulation vessel and was operated at 6 to 10 kRPM for 5-15 minutes. This process resulted in a homogenous micro-emulsion (ME) suspension of squalene emulsion particles in the 4 to 20 μm diameter range which were suitable for additional size reduction by microfluidization in a high-pressure homogenizer to create a squalene nanoemulsion (SNE).

### Fine Homogenization to Produce the Squalene Nanoemulsion (SNE)

After coarse homogenization, the emulsion was further processed using a high-pressure homogenizer/microfluidizer to produce nanometer-sized emulsion particles. The ME was introduced to a high-pressure homogenizer such as the Microfluidics low volume Microfluidizer®, the GEA Group PandaPlus 2000 or Bee International, NanoDeBEE and a recirculation loop is established. A counter-flow heat exchanger, fed by a Controlled Temperature Unit with a set point of 5° C., is included in the recirculation loop to neutralize the heat generated through high pressure homogenization. For the production of emulsion particles of desired size and processability, 20 kPSI was selected as the operating set point for this process step. The high-pressure homogenizer operates at a constant and unalterable flow rate through the established recirculating loop. Using this measured flow rate and the volume of ME to be processed, the theoretical time required for the entirety of the formulation to make a single pass through the recirculation loop was calculated. Given this calculated single pass time, the high-pressure homogenizer was usually operated until the desired pass count of at least 10 was reached, yielding either the SNE or CLA-SNE.

### Filtration

After formulation, the SNE or CLA-SNE was passed through a 0.8/0.2 μm PES filter. A flux of 42 LMH through the filter was selected given its optimal mass yield and particle stability through filtration.

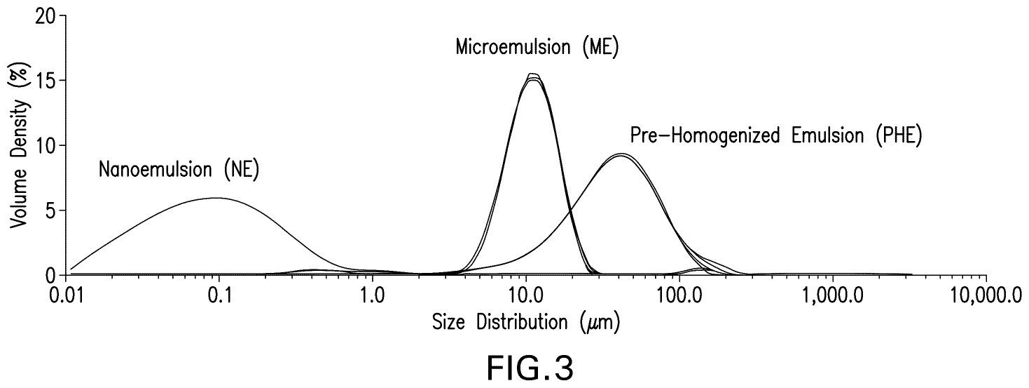

A laser diffraction or static light scattering (SLS) technique using a Malvern Panalytical Ltd. MS3000 instrument was utilized to measure the volume-weighed size distribution of a nanoemulsion during preparation. This data was then analyzed to calculate the size of the particles that created the scattering pattern. Sample fractions of pre-homogenized emulsion (PHE), micro-emulsion (ME), and squalene nanoemulsion (SNE) were obtained. These emulsion formulations were diluted to target an obscuration of 3% into 5 mM Histidine pH 5.8 and 2.5 mM NaCl buffer and SLS was performed and collected under recirculation of 1200 RPM. Sample data sets were collected with a scan of 30 seconds per data set. Three data sets from each step in the CLA-SNE formulation process are summarized in FIG. 3. Although 20 mM Histidine pH 5.8 buffer was a perfectly suitable formulation for the stability of the bulk during process and when stored in polymeric containers (e.g., plastic), upon storage in glass, non-specific absorption of the CLA-SNE or SNE to the surface of the glass was observed. A screen evaluating surfactants/solubilizers, buffers and salts was evaluated, and multiple formulations show success in eliminating this stability issue with the selected formulation of 20 mM Histidine 0.05% PS-20 and 75 mM NaCl being selected as the stabilizing formulation (data not shown).

### Example 2: Preparation of a Squalene Nanoemulsion (SNE) Adjuvant System and Addition of the Cationic Lipid, (13Z,16Z)-N,N-dimethyl-3-nonyldocosa-13,16-dien-1-amine) or CLA as Free Base Directly After Microfluidization of the SNE

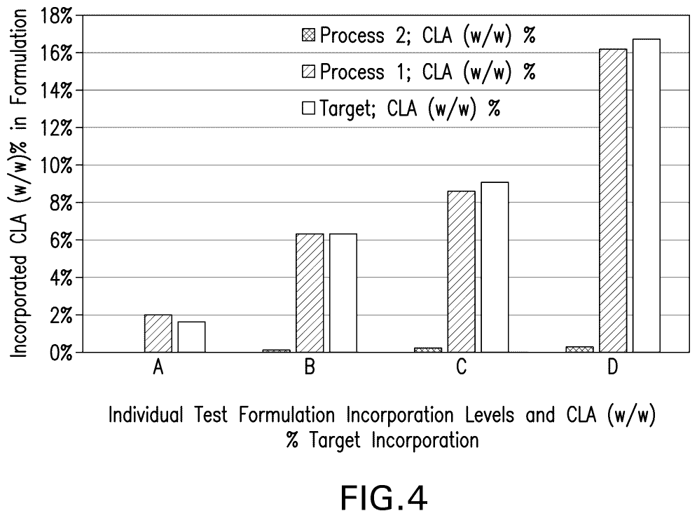

Two formulation processes were evaluated for incorporating CLA into the nanoemulsion particle which includes PS-20, sorbitan trioleate (SPAN-85) and squalene formulated in Histidine pH 5.8 buffer. In the first process (referred to as Process 1), the SNE was prepared using the process described in Example 1. In the second process, (referred to as Process 2), only PS-20, sorbitan trioleate (SPAN-85) and squalene were combined and mixed together. Once mixed and blended, a histidine buffer was added and mixed with the initial emulsion components (PS-20, sorbitan trioleate and squalene). Blended emulsion components were first subjected to course homogenization to produce the microemulsion ME followed by microfluidization to produce the nanoemulsion (NE), as described in Example 1. In a separate glass vessel, 0.25 mg/mL CLA was dissolved in 100% ethanol at room temperature. A sufficient volume of this CLA ethanol solution, to produce the desired final CLA concentration, was then added to the SNE containing PS-20, sorbitan trioleate and squalene in Histidine buffer pH 5.8 and then mixed for 60 minutes at room temperature. After incubation, the formulation was then dialyzed against 5 mM histidine 2.5 mM NaCl pH 5.8 at 10 mL sample to 500 mL buffer over night at 4° C., with two buffer changes. The two processed emulsions (Process 1 and 2) were then examined for CLA incorporation into the SNE using UPLC-CAD. The results indicate that no significant quantity of CLA was incorporated as compared to the individual formulation (w/w) % target incorporation using Process 2 which indicates that Process 1 is preferred for the successful incorporation and stability of CLA in the nanoemulsion (FIG. 4).

### Example 3: Impact of Time and Temperature on Nanoemulsion Formulation Physical Stability Using NTA and DLS

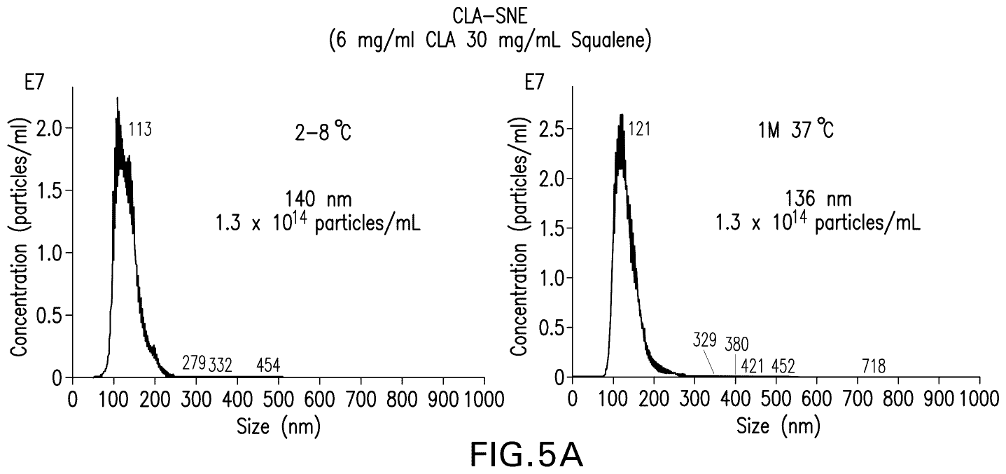

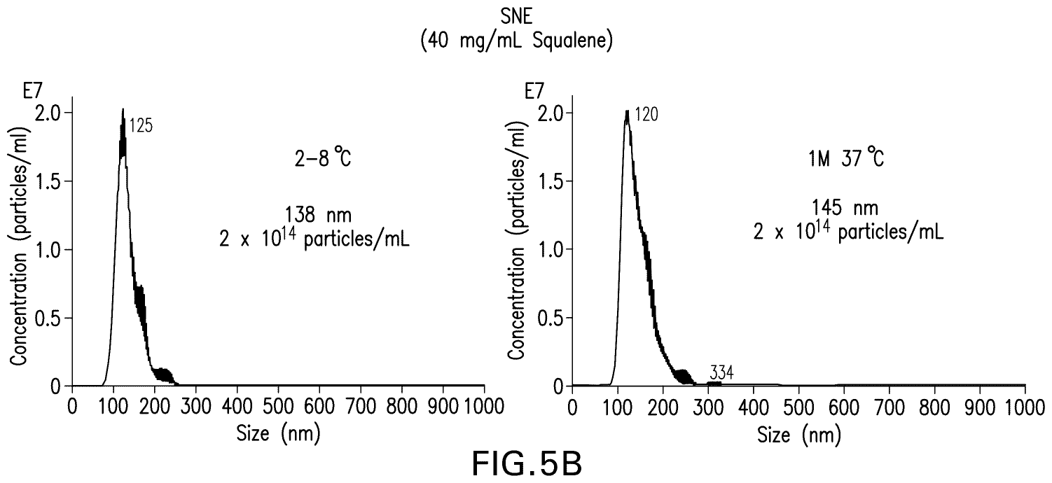

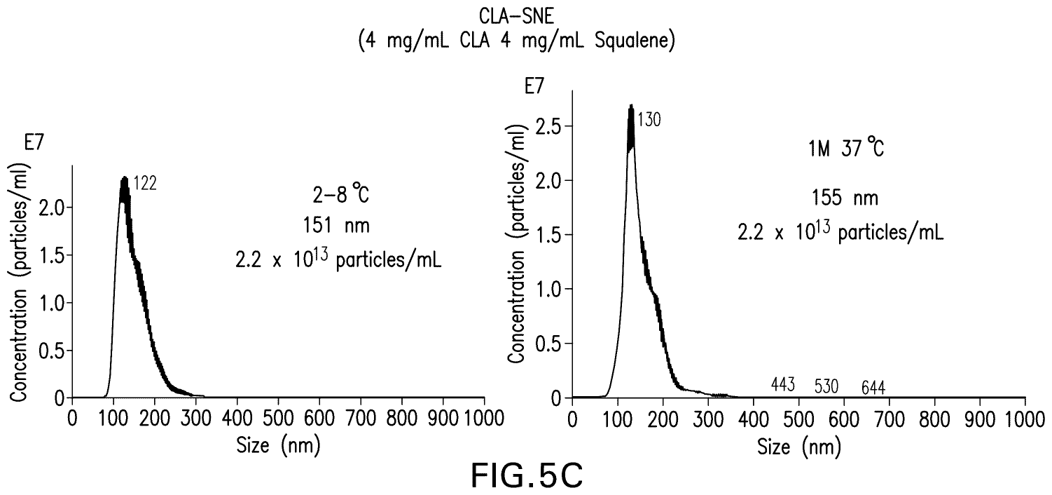

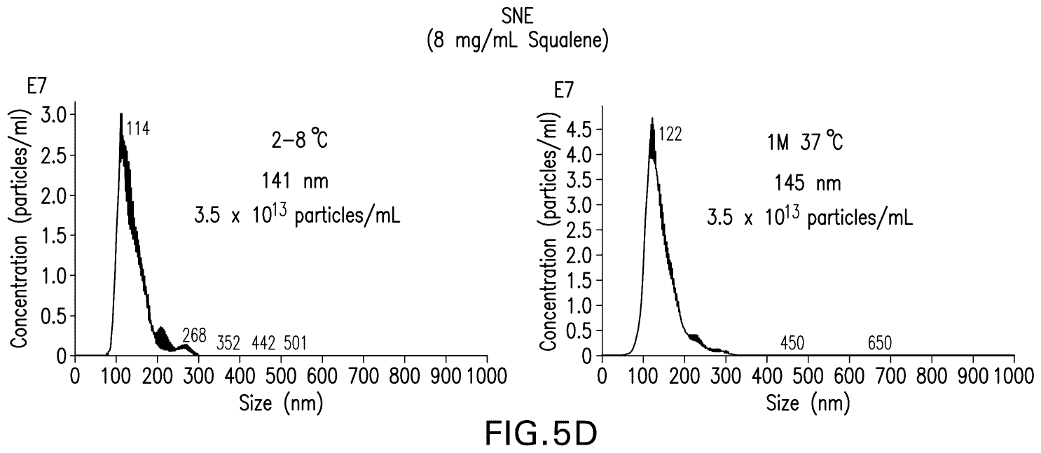

As shown in FIGS. 5A-5D, to assess stability of the nanoemulsion systems (CLA-SNE or SNE), prepared as described in Examples, supra, nanoparticle tracking analysis (NTA) was utilized. The technique collects videos of directly tracked nanoparticle populations as they move by Brownian motion to extrapolate particle size and concentration. A class 1, 635 nm laser focuses an 80 mm red laser beam through the liquid sample, illuminating particles as rapidly diffusing points of light. A CCD camera records a 30 frame per second video to track the movement of each individual illuminated particle over time. The system software identifies the center of each individual particle from the video and tracks the distance independently traversed to determine the mean square displacement. This tracking was performed simultaneously for every particle within the sample population in each frame until the raw data collected from the entire video was analyzed. By simultaneously measuring the mean square displacement of every individual particle tracked, its diffusion coefficient (Dt) and the spherical equivalent hydrodynamic radius (rh) were determined by applying the Stokes-Einstein equation. The software then represents this accumulated data as a particle size and concentration distribution. Raw data information on not only particle size and concentration, but also intensity, or brightness of the individual particle were gathered. Taken together the data were fitted and plotted individually as particle intensity relative to particle size, and particle concentration relative to particle size, and then on three-dimensional contour plots comparing particle size, concentration, and intensity of all particle populations.

Upon exposure of the nanoemulsion formulations (CLA-SNE or SNE) to 37° C. for up to 1 month, no significant change in particle concentration or size distribution of the nanoemulsions was observed as evaluated using NTA (FIG. 5).

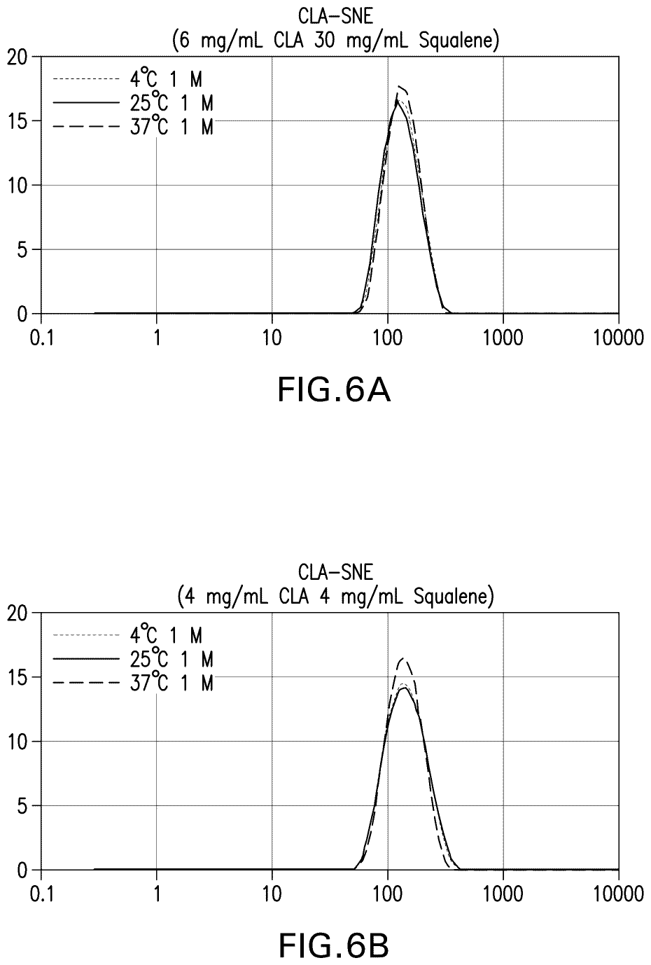

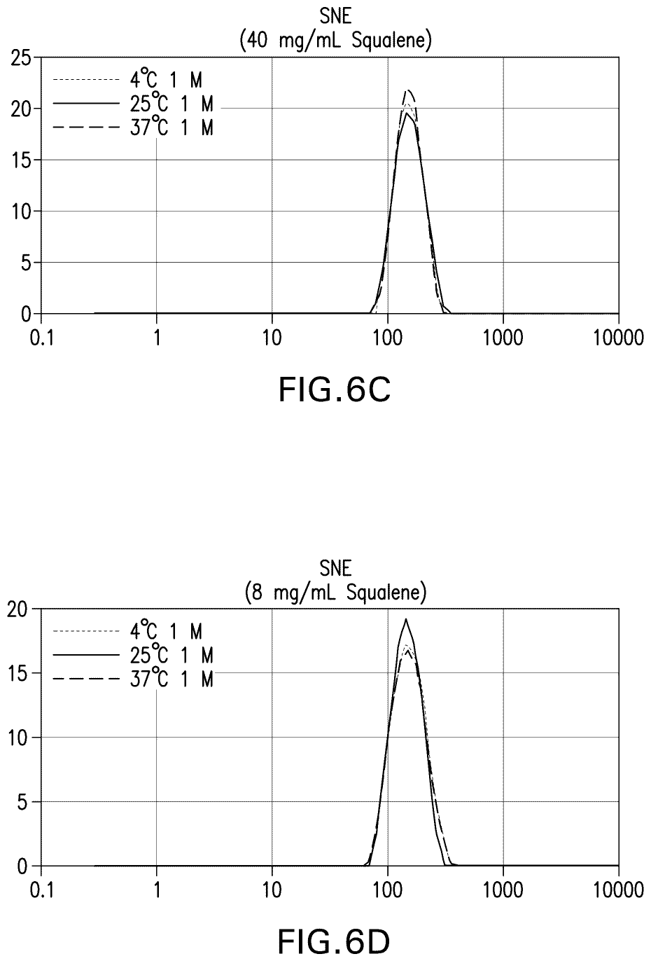

A nanoemulsion may be susceptible to aggregation within the 10-1000 nm particle size range, thereby making DLS a suitable stability indicating technique for assessing and quantitating aggregation phenomena. To assess stability of the nanoemulsion systems, prepared as described in examples, supra, dynamic light scattering (DLS) was utilized to measure the average particle size distribution. DLS instruments use a laser to illuminate particles in a solution and then examine the changes in intensity of the scattered light over time as a result of Brownian motion. The correlation of the scattered light intensity over time to the intensity at time zero results in an exponential decay curve or correlation function. The rate of decay in the correlation function with respect to time is much faster for smaller particles than larger particles and this forms the basis for calculation of the particle sizes. Upon exposure of the nanoemulsion to 4° C., 25° C. or 37° C. for up to 1 month, no change in the size distribution of the nanoemulsions was observed by DLS (FIGS. 6A-6D). The Z-average remained around 110 nm to 180 nm for the CLA-SNE or SNE.

### Example 4: Impact of Time and Temperature on Nanoemulsion Formulation Chemical Stability Using UPLC-CAD

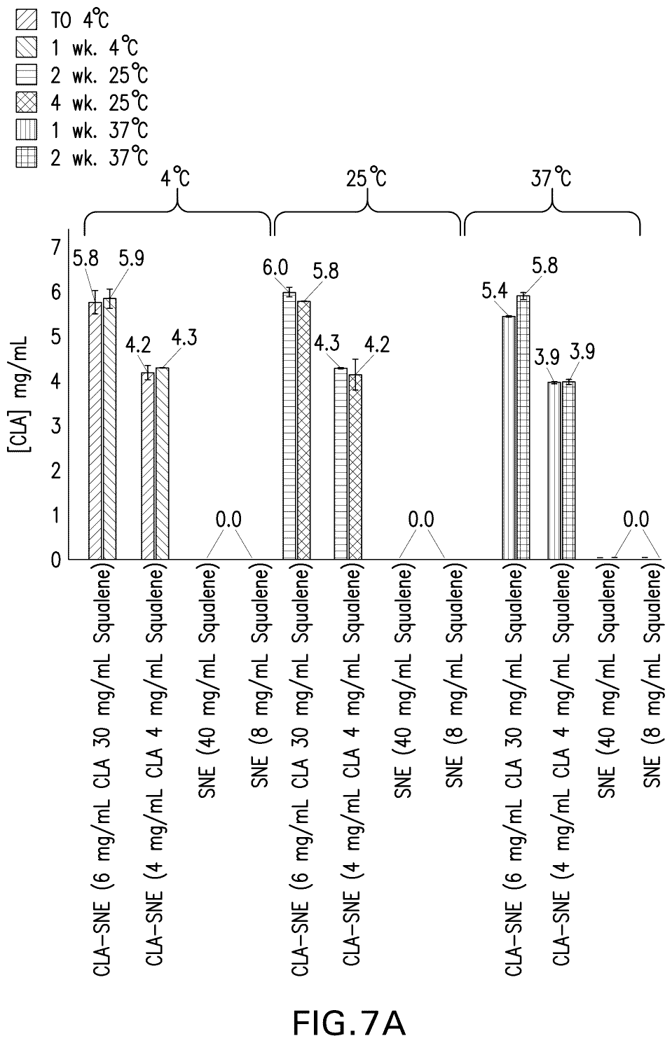

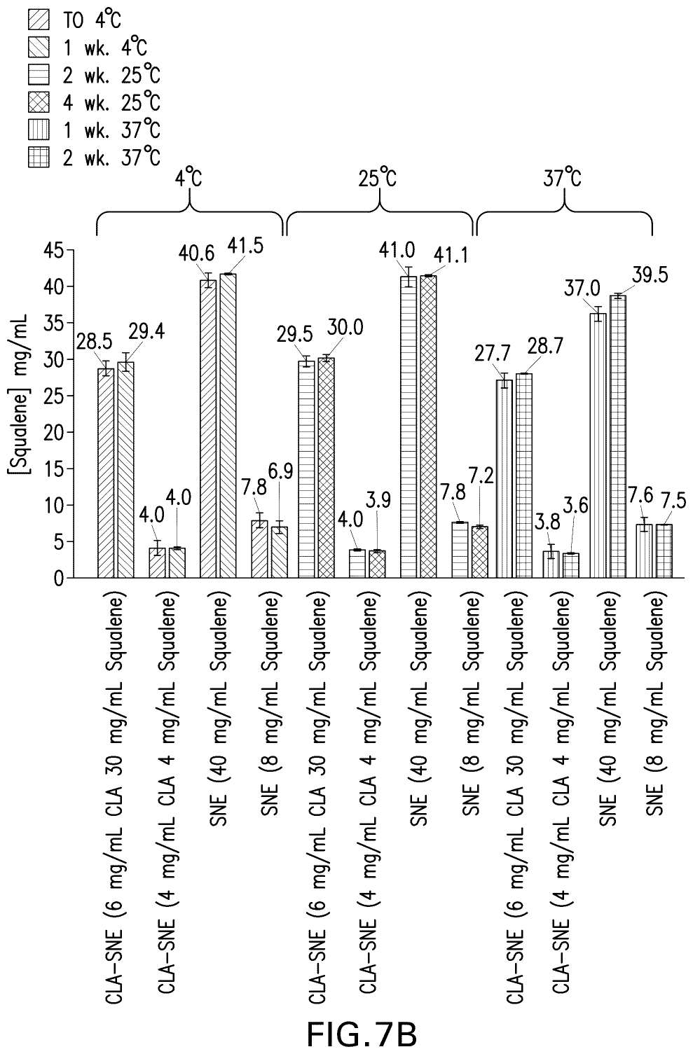

To assess the chemical stability of the nanoemulsion systems, prepared as described in the Examples, supra, ultra-performance liquid chromatography coupled with a charge aerosol detector (UPLC-CAD) was utilized to measure the stability of the CLA (CLA-SNE only) and squalene concentration upon storage at 4° C., 25° C. and 37° C. for 1 month. Upon exposure of the nanoemulsion to 4° C., 25° C. and 37° C. for up to 1 month, the concentration of CLA (FIG. 7A) or squalene (FIG. 7B) in the SNE was not impacted, demonstrating excellent chemical stability in both CLA-SNE and SNE adjuvants.

Moreover, UPLC-CAD can quantitate the production of degradation products because of chemical breakdown of either squalene or CLA. Upon exposure of the SNE or CLA-SNE adjuvant systems to elevated temperature, no detectable degradation peaks were observed indicating that the squalene and CLA components of CLA-SNE and SNE adjuvant systems have excellent thermal stability (data not shown).

### Example 5: Preparation of a Squalene Nanoemulsion (SNE) Adjuvant System with and without the Cationic Lipid, (13Z,16Z)-N,N-dimethyl-3-nonyldocosa-13,16-dien-1-amine) by Microfluidic Nanoemulsion Self-Assembly (MNS)

The squalene nanoemulsion adjuvant is prepared with and without the ionizable cationic lipid (13Z,16Z)-N,N-dimethyl-3-nonyldocosa-13,16-dien-1-amine), also referred to as CLA (FIG. 1). The microfluidic nanoemulsion self-assembly (MNS) process used to prepare an SNE. The SNE is multi-component emulsion formulation consisting of 3 stabilizing ingredients; squalene, sorbitan trioleate (SPAN-85), and polysorbate-20 (PS-20) with CLA (referred to as CLA-SNE, Table IV) or without a CLA (referred to as SNE, Table V). The biophysical characteristics (e.g., particle size, chemical compositions) and stability of MNS-prepared-SNE/CLA-SNE formulations are very similar to that of the high pressure fine homogenization process for preparation of SNE/CLA-SNE formulations described in Example 3. Essentially, the microfluidic nanoemulsion self-assembly (MNS) process described in this example is an alternative process for preparing a squalene nanoemulsion (SNE) adjuvant system. The nanoparticle self-assembly process described in this example was conducted using a “microfluidics” ethanol/aqueous mixing instrument. However, the ethanol/aqueous stream nanoparticle self-assembly process described in this invention is not limited by “microfluidic” mixing. Mixing larger volume streams of hydrophobic solvents with aqueous solutions can be accomplished using a Tec-mixing process outlined in Example 4.

SNE MNS formulations can generally be prepared by dissolving the cationic lipid, squalene, SPAN-85, and PS-20 at the targeted concentrations into an appropriate non-aqueous solvent such as ethanol. The self-assembly procedure involves combining a stream of the ethanol dissolved hydrophobic emulsion components with a stream of the aqueous emulsion solution. As the two solvent streams combine, the hydrophobic molecules (i.e., the cationic lipid, squalene, SPAN-85, and PS-20) interact with the aqueous solvent. The molecules then assemble themselves into an emulsion of nanosized particles, as described below. Following the formation of the self-assembled emulsion of nanoparticles, the residual ethanol can be removed from the squalene emulsion by several suitable means. In this example, the ethanol was reduced to less than 0.1% (w/v) by overnight dialysis with the aqueous buffer. The resulting SNE formulation was sterilized by filtration through a 0.2 μm pore size sterilization filter. Several process parameters within each step, such as order or addition, mixing times, temperature, concentration of non-aqueous components, concentrations of aqueous buffer components, aqueous pH, non-aqueous to aqueous solution mixing ratio, total flow rate, and waste discard volumes were controlled to yield SNE adjuvant systems with the desired attributes.

TABLE IV
Composition of CLA-SNE Adjuvant Prepared by MNS

| | | Content of | Molecular | Content of |
|---|---|---|---|---|
| | | Each Lipid | Weight | Each Lipid |
| Component | Description | (Mole %) | (g/mol) | (Mass %) |
| CLA | (13Z,16Z)-N,N- | 4-45% | 475.9 | 4-46% |
| | dimethyl-3- | | | |
| | nonyldocosa- | | | |
| | 13,16-dien-1-amine | | | |
| Squalene | squalene | 52-89% | 410.72 | 45-80% |
| SPAN-85 | sorbitan trioleate | 2-4% | 957.5 | 4-8% |
| PS-20 | polysorbate-20 | 2-3% | 1228 | 4-8% |

| | | |
|---|---|---|
| Buffer | 20 mM Histidine, | N/A |
| Matrix | pH 5.8 | |

TABLE V
Composition of SNE Adjuvant Prepared by MNS

| | | Content of | Molecular | Content of |
|---|---|---|---|---|
| | | Each Lipid | Weight | Each Lipid |
| Component | Description | (Mole %) | (g/mol) | (Mass %) |
| Squalene | squalene | 92.9% | 410.72 | 83.3% |
| SPAN-85 | sorbitan trioleate | 4.0% | 957.5 | 8.3% |
| PS-20 | polysorbate-20 | 3.1% | 1228 | 8.3% |

| | | |
|---|---|---|
| Buffer | 20 mM Histidine, | N/A |
| Matrix | pH 5.8 | |

### Formulation Preparation Using Microfluidic Nanoemulsion Self-Assembly

In this example, one SNE and four CLA-SNE formulations were prepared by the microfluidic nanoemulsion self-assembly procedure in 20 mM histidine pH 5.8 for biophysical characterization. The self-assembled nanoemulsion process starts with 15 mg/mL squalene, 1.5 mg/mL SPAN-85 and 1.5 mg/mL PS-20 completely dissolved in ethanol. In addition, each of the ethanol solutions described above also contained CLA at either 0.75, 1.5, 5.0 or 15.0 mg CLA/mL. Thus, the initial “target” CLA/squalene (w/w) % for all five formulations would be 0.0, 5.0, 10.0, 33.3, and 100 (w/w) % CLA/squalene. The aqueous buffer for all the formulations was 20 mM histidine at pH 5.8. A benchtop NanoAssemblr™ instrument from Precision NanoSystems, Inc. (Vancouver, BC, Canada) was used to self-assemble the one SNE and four CLA-SNE adjuvant nanoemulsions

The self-assembled squalene nanoparticle formulations were prepared in the following manner. A 1 mL syringe was filled with a little over 0.7 mL of the hydrophobic compound mixture dissolved in ethanol, while a 3 mL syringe was filled with a little over 1.4 mL of the aqueous 20 mM histidine pH 5.8 buffer. After both syringes were loaded with the appropriate amount of solution, the syringes were attached to the NanoAssemblr™ instrument. The following microfluidic mixing parameters were programed into the NanoAssemblr™: a) Total volume=2 mL, b) Flow rate ratio=2:1 (aqueous to ethanol), c) Total Flow Rate=12 mL/min, d) Start waste volume=0.25 mL, and c) End waste volume=0.05 mL. The instrument was activated to start the ethanol and aqueous solution mixing process in as little as a few seconds. Approximately 2.0 mL of post-mixing nanoparticle emulsion in approximately 30% ethanol was collected in a 15 mL Falcon tube for each of the 5 formulations. A fresh NanoAssemblr™ mixing cartridge was used for each of the 5 different SNE and CLA-SNE formulations described above. The ethanol concentration was reduced in each formulation by overnight dialysis. After dialysis, all of the samples were stored at 4° C. prior to analytical characterization.

### Analytical Characterization

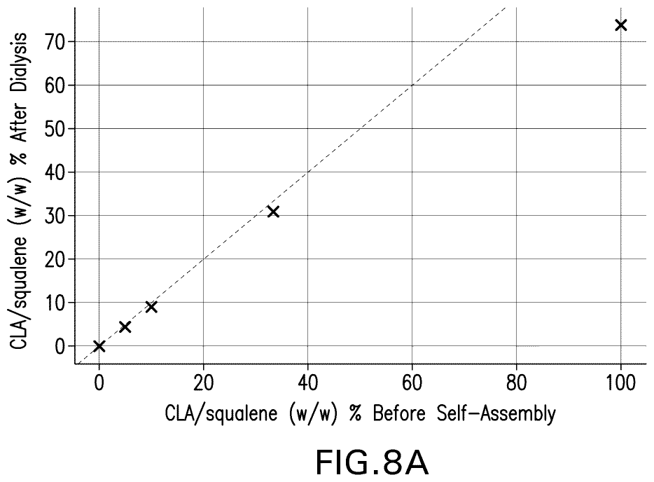

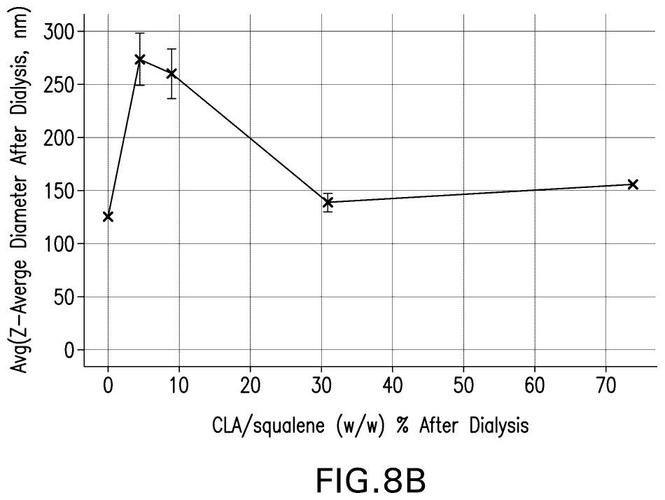

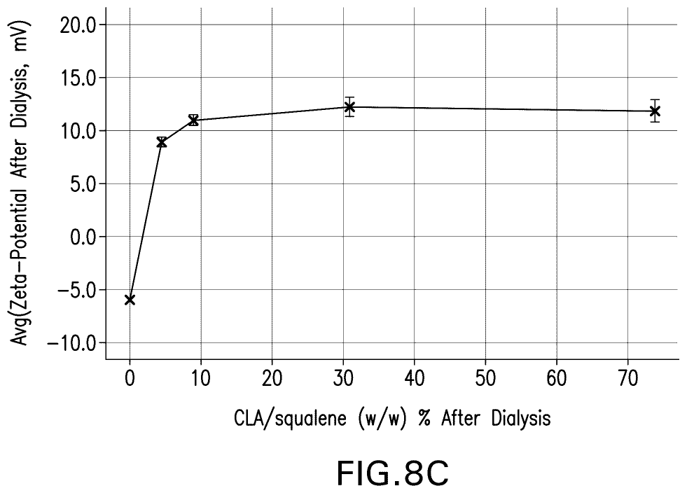

The cationic lipid, CLA, and squalene were equally incorporated into the squalene CLA-SNE nanoparticles prepared by MNS as shown in FIG. 8A. The CLA/squalene (w/w) % ratio after dialysis (i.e., the y axis) to remove the process ethanol was compared to the CLA/squalene (w/w) % before self-assembly (i.e., the x axis) while in the ethanol solution for all the formulation samples described in this example. The “measured” CLA/squalene (w/w) % after MNS and dialysis was equal to the “target” (w/w) % up to at least 35 (w/w) %. Even at a 100% “target” CLA/squalene (w/w) % prior to self-assembly over 70% of the available CLA was incorporated into the CLA-SNE nanoparticles relative to the squalene content in the MNS prepared nanoparticle emulsion. The CLA/squalene (w/w) % ratios were measured by reverse phase UPLC-CAD. CLA was clearly incorporated into CLA-SNE by prepared by microfluidic nanoemulsion self-assembly (MNS) process.

The intensity weighted Z-average DLS diameters of the CLA-SNE formulations prepared the MNS process were measured using a Malvern ZetaSizer Ultra. Aliquots of post-dialyzed CLA-SNE samples from each formulation were diluted at either 50- or 100-fold in 2.0 mL 20 mM histidine pH 5.8 buffer. Average DLS diameter and standard deviation was plotted versus the measured post-dialysis CLA/squalene (w/w) % for each formulation and is shown in FIG. 8B. Three DLS measurements were made at room temperature for each formulation. The standard deviation bars are show unless the standard deviation is less that data point image. The intensity weight Z-average DLS diameters of MNS prepared CLA-SNE ranged from approximately 150 to 280 nm which is similar to CLA-SNE nanoparticles prepared by high-pressure homogenization. Varying MNS process parameters such as those described above in this example were controlled to yield CLA-SNE adjuvant systems with the desired diameters. The measured Zeta Potential of CLA-SNE squalene nanoparticle formulations at pH 5.5 prepared the MNS process are shown in FIG. 8C. The Zeta Potential was measured using a Malvern ZetaSizer Ultra. Aliquots of post-dialyzed CLA-SNE samples from each formulation were diluted at either 50 or 100× in 2.0 mL of 20 mM citrate BIS TRIS propane buffer at pH 5.5. Three Zeta potential measurements were made at room temperature for each formulation. The standard deviation bars are show unless the standard deviation is less that data point image. The Zeta Potential of the 0 (w/w) % CLA CLA-SNE formulation, i.e., no CLA, was around-5 mV. As illustrated in FIG. 8C, addition of CLA significantly increased the nanoparticle Zeta Potential to around +10 mV.

### Example 6: Optimization of CLA-SNE Preparation by Alteration of the Aqueous Phase pH

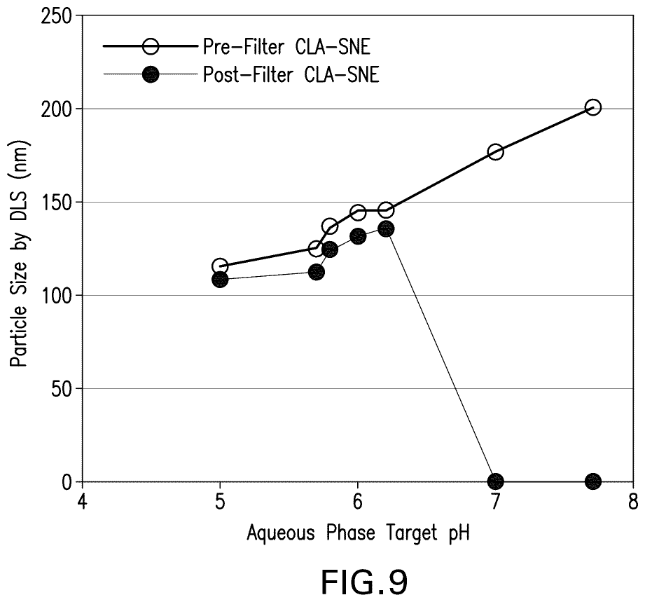

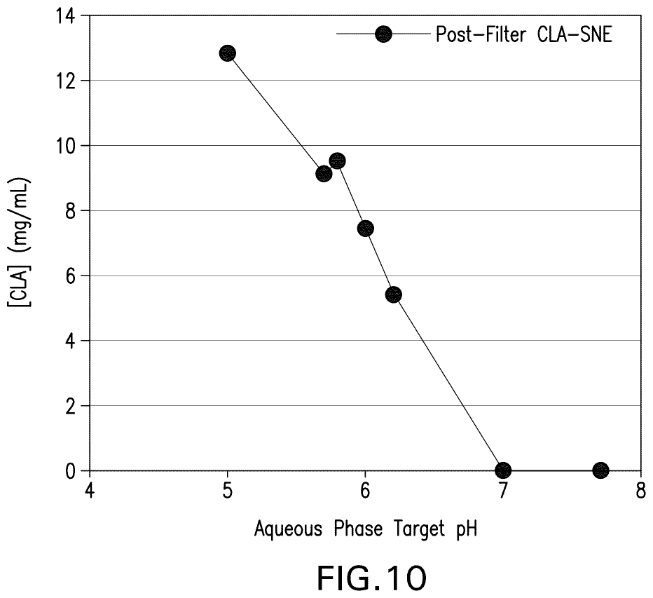

The CLA-SNE process involves the use of a reversible cationic CLA molecule with an observed pKa of 6.4. Addition of CLA to the SNE preparation process and final matrix with a pH of 5.8 results in the protonation of CLA, which functions to give an overall net positive charge to CLA-SNE particles as well as any intermediates of the preparation process. CLA-SNE preparation culminates in a 0.8/0.2 μm filtration event, a process step which had proved difficult to perform, with significant filter fouling and low product yield consistently observed. However, the filtration of SNE did not demonstrate the same magnitude of these filtration challenges and was a more efficient nanoemulsion filtration step. Importantly, without CLA present SNE docs not carry the strong positive charge observed with CLA-SNE. In an effort to prepare an uncharged CLA-SNE for a higher efficiency CLA-SNE filtration step, a series of experiments were performed in which the pH of the aqueous phase (20 mM L-Histidine) was adjusted prior to use in the preparation of CLA-SNE. 20 mM L-Histidine was prepared with pH targets of 5.0, 5.7, 5.8, 6.0, 6.2, 7.0, and 7.7. Each buffer was then used as the aqueous phase during the CLA-SNE preparation process with a target formulation target of 15 mg/mL CLA, CLA-SNE preparation proceeded exactly as described in Example 3. Upon completion of homogenization process step, the particle size of the CLA-SNE intermediate was measured by DLS using a Malvern Panalytical Nano ZS. Filtration with 0.8/0.2 μm PES filter was then performed. Particle size distribution was measured post-filtration by DLS and [CLA] was quantified by UPLC-CAD. For CLA-SNE samples prepared with 20 mM L-histidine with pH 7.0 or 7.7, complete filter fouling was observed immediately upon application of material to the filter and no collection of filtered material was possible making quantification by DLS or UPLC-CAD impossible, values of 0 are reported for illustration purposes. In pre-filtered samples, a trend was observed of increasing particle size by DLS as the pH of the aqueous phase buffer was increased (FIG. 9). This relationship was maintained in post-filter samples with each CLA-SNE sample demonstrating a modest reduction in particle size post-filtration, with the obvious exception of pH 7.0 and 7.7 samples which again demonstrated complete filter fouling immediately and no material recovery was possible. For post-filtered samples, it was observed that [CLA] decreases in the final CLA-SNE material as the pH of the aqueous phase is increased (FIG. 10). This relationship demonstrates that CLA-SNE preparation using an aqueous phase at a lower pH results in an increase in process yield in the terminal filtration and a more efficient SLA-CAN manufacturing process.

### Example 7: Immunogenicity and Durability of a Two-Dose of 9vHPV Vaccine+CLA-SNE (0.33 mg, 1.32 mg, 3.96 mg) or SNE (2.16 mg) Adjuvant in Rhesus Macaques

The objective of study SD-HPV-062 was to evaluate the immunogenicity of 9vHPV Vaccine (9vHPV+AAHS) when combined with increasing doses of CLA-SNE adjuvant (0.33 mg, 1.32 mg, or 3.96 mg (total lipid)) or SNE (2.16 mg (total lipid)) in a nonhuman primate nonclinical immunogenicity model. The group designations are described in Table VI. At Week 0, groups of 4-5 rhesus macaques were inoculated with either 9vHPV Vaccine alone or with 9vHPV Vaccine combined with 0.33 mg, 1.32 mg or 3.96 mg of CLA-SNE or 2.16 mg of SNE adjuvants. At Week 24, all groups were given a second dose of 9vHPV Vaccine with the respective adjuvants and dose. The 1.0-mL doses were prepared by mixing 9vHPV Vaccine and 0.5 mL of a 2× concentration CLA-SNE or SNE adjuvants and administering into the rhesus macaque quadricep (0.5 mL×2 quads/NHP) within 1 hour of formulation.

TABLE VI
Groups, Dose Levels, and Dosing Schedule in Non-Human Primates

| | No. of | | | | | | |
|---|---|---|---|---|---|---|---|
| | rhesus | | Dose level | | | | Dosing |
| Group | macaques | Inoculum | 9vHPV Vaccine ^{b} | CLA-SNE | SNE | ROAª | schedule |
| 1 | 4 | 9vHPV Vaccine | 1/20^{th} human | — | — | IM | Weeks |
| | | | dose ^{b} | | | | 0, 24 |
| 2 | 5 | 9vHPV Vaccine + | | 3.96 mg^{c} | — | IM | Weeks |
| | | CLA-SNE | | | | | 0, 24 |
| 3 | 5 | 9vHPV Vaccine + | | 1.32 mg^{d} | — | IM | Weeks |
| | | CLA-SNE | | | | | 0, 24 |
| 4 | 5 | 9vHPV Vaccine + | | 0.33 mg^{e} | — | IM | Weeks |
| | | CLA-SNE | | | | | 0, 24 |
| 5 | 4 | 9vHPV Vaccine + | | — | 2.16 mg^{f} | IM | Weeks |
| | | SNE | | | | | 0, 24 |

9vHPV = human papillomavirus 9-valent vaccine;
IM = intramuscular;
NA = not applicable;
ROA = route of administration.
ªAll doses were delivered in 0.5 ml × 2 quadriceps. 0.5 ml 9vHPV vaccine diluted in PBS; 0.5 ml 2X adjuvant final fill container (AFC)
^{b}One rhesus monkey dose of 9vHPV Vaccine is equivalent to 1/20 of one human dose of 9vHPV vaccine.
^{c}CLA-SNE = 3.6 mg L608/mL; 3.6 mg Squalene/mL; 0.36 mg PS-20/mL; 0.36 mg SPAN-85/mL − 7.92 mg Total Lipid/mL = 2X concentration
^{d}CLA-SNE = CLA-SNE AFC; 1.2 mg L608/mL; 1.2 mg Squalene/mL; 0.12 mg PS-20/mL; 0.12 mg SPAN-85/mL − 2.64 mg Total Lipid/mL = 2X concentration
^{e}CLA-SNE = CLA-SNE AFC 0.3 mg L608/mL; 0.3 mg Squalene/mL; 0.03 mg PS-20/mL; 0.03 mg SPAN-85/mL − 0.66 mg Total Lipid/mL = 2X concentration
^{f}SNE = 3.6 mg Squalene/mL; 0.36 mg PS-20/mL; 0.36 mg SPAN-85/mL − 4.32 mg Total Lipid/mL = 2X concentration

Rhesus macaques (n=4-5/group) were injected intramuscularly with two (Week 0 and Week 24) doses of 9vHPV Vaccine or two (Week 0 and Week 24) doses of 9vHPV Vaccine combined with either 3.96, 1.32, 0.33 mg CLA-SNE or 2.15 mg SNE. Antibody levels against all 9 HPV VLP types were monitored for 54 weeks. To assess immunogenicity, sera from individual animals were evaluated using a multiplex assay for antibody levels to all 9 HPV types in the vaccine. VLP-specific HPV antibody concentrations were determined at study weeks 0, 4, 6, 8 12, 20, 24, 26, 28, 30, 32, 34, 36, 44 and 54.

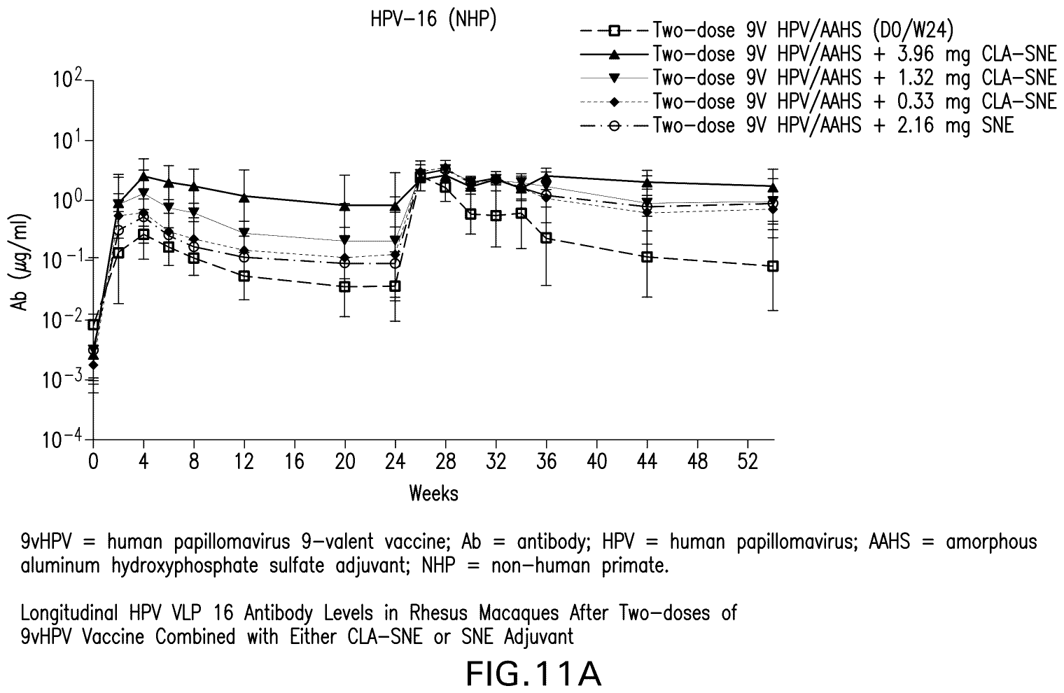

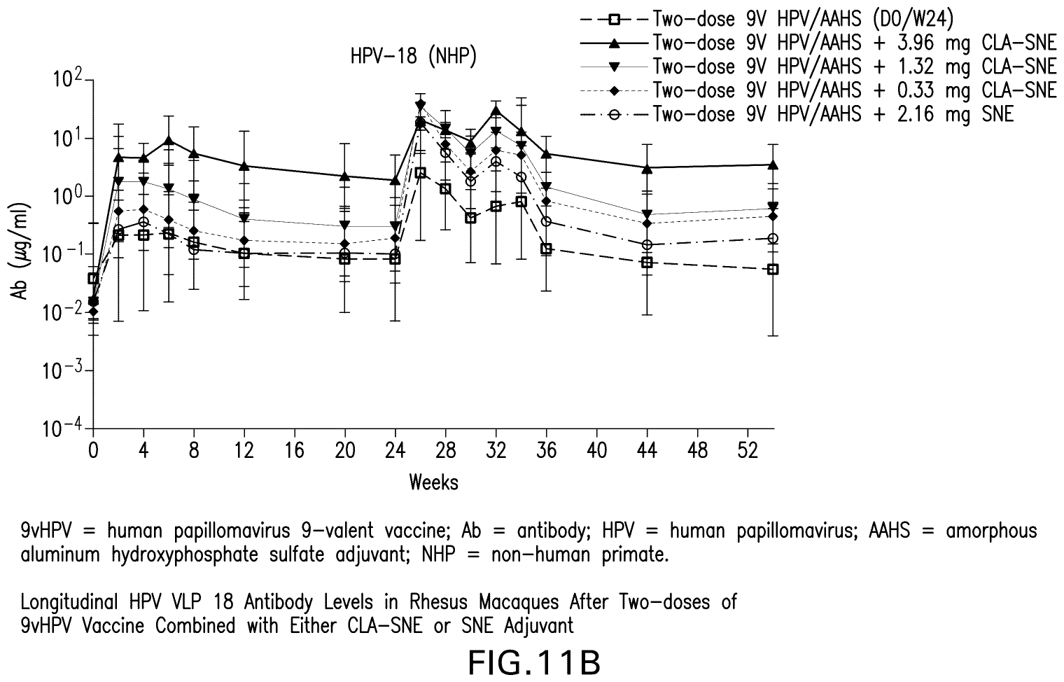

Representative titers to HPV VLP-16 and HPV VLP-18 are shown ins FIGS. 11A and 11B. The antibody concentrations (μg/mL) detected in serum against HPV VLP type 16 (FIG. 1A) and 18 (FIG. 1B) at weeks 0, 4, 6, 8 12, 20, 24, 26, 28, 30, 32, 34, 36, 44 and 54. The data are presented as geometric mean concentrations and 95% confidence intervals.

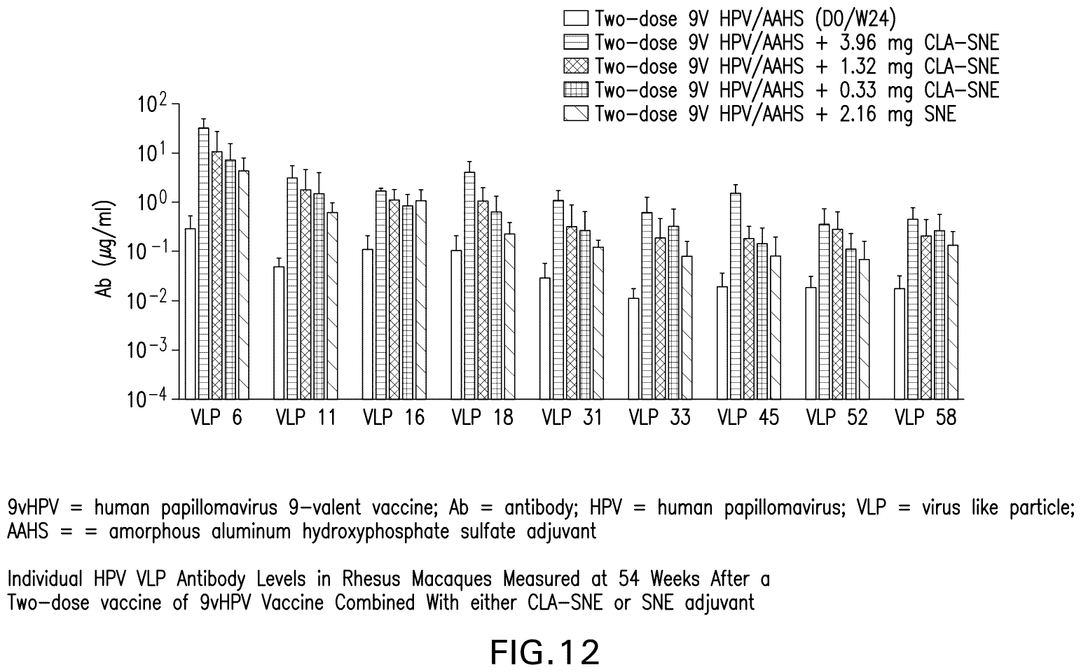

As shown from the figures, the adjuvanting effect of CLA-SNE on HPV antibody titers was clearly dose dependent. For SNE, one dose was tested to match the oil in water emulsion concentration of the highest dose of CLA-SNE. Animals inoculated with 9vHPV Vaccine combined with any CLA-SNE or SNE dose yielded HPV16 antibody titers higher than the two-dose 9vHPV Vaccine group at all timepoints except week 24/week 26 when the HPV16 titers achieve maximal titers for all groups. Animals inoculated with 9vHPV Vaccine combined with any CLA-SNE dose yielded HPV18 antibody titers higher than the two-dose 9vHPV Vaccine group at all timepoints. Animals inoculated with 2.16 mg SNE did not have higher HPV18 titers after post-dose 1 but did show geomean titers higher at longitudinal time points post-dose 2. Importantly, these titers remained higher than in the two-dose control group throughout the end of the study (54 weeks). As shown in FIG. 12, rhesus macaques (n=4-5/group) were injected intramuscularly with two (Week 0 and Week 24) doses of 9vHPV Vaccine or two (Week 0 and Week 24) doses of 9vHPV Vaccine combined with either 3.96, 1.32, 0.33 mg CLA-SNE or 2.16 mg SNE. Antibody levels against all 9 HPV VLP types were monitored for 54 weeks. Shown are the antibody concentrations (μg/mL) detected in serum against the 9 VLP types at week 54. The data are presented as geometric mean concentrations and 95% confidence intervals.

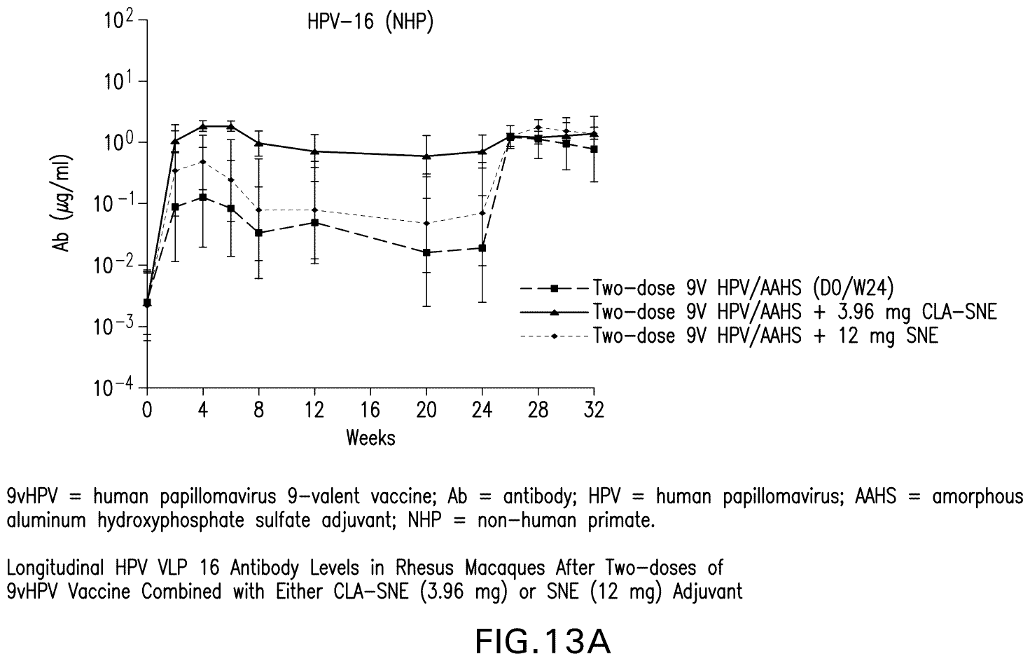

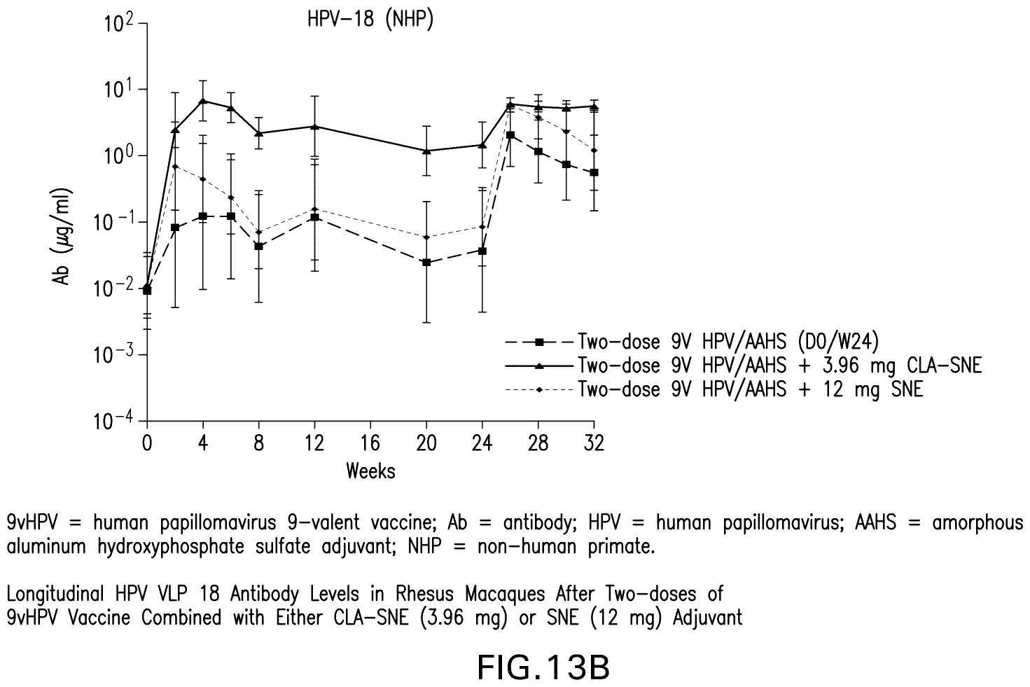

To assess immunogenicity, sera from individual animals were evaluated using a multiplex assay for antibody levels to all 9 HPV types in the vaccine. VLP-specific HPV antibody concentrations were determined at study weeks 0, 4, 6, 8 12, 20, 24, 26, 28, 30, and 32. Data is being collected for weeks 36, 44 and 54. Representative titers to HPV VLP-16 and HPV VLP-18 are shown in FIGS. 13A and 13B. Rhesus macaques immunized with 9vHPV Vaccine plus 3.96 mg of VA-879 CLA-SNE achieved peak antibody titers for HPV16 and HPV18 at 4 weeks post-dose 1 and remained very stable until boosting at 24 weeks, where again the titers remained very high and stable through week 32. Rhesus macaques immunized with 9vHPV Vaccine plus 12 mg of VA-881SNE showed geomean antibody titers consistently higher than one dose 9vHPV and similar or higher titers for HPV16 and HPV18 after the second dose of all vaccines at week 24. These results show that increasing the amount of VA-881SNE to 12 mg total lipid (10 mg squalene) results in higher geomean antibody titers for HPV VLPs.

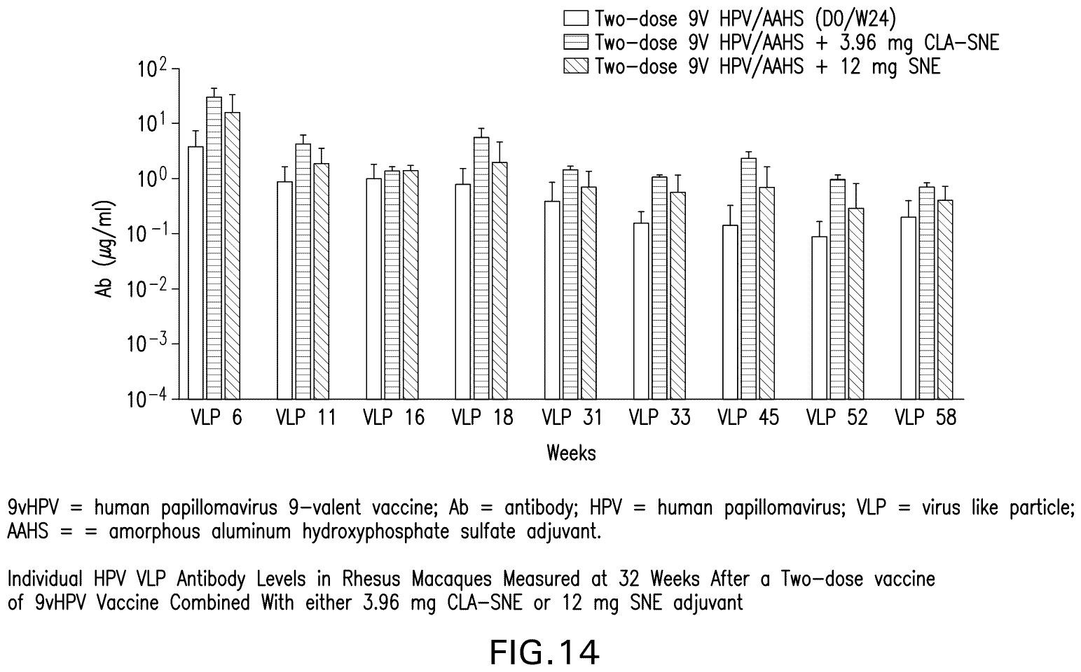

Rhesus macaques (n=5/group) were injected intramuscularly with two (Week 0 and Week 24) doses of 9vHPV Vaccine or two (Week 0 and Week 24) doses of 9vHPV Vaccine combined with either 3.96 mg CLA-SNE or 12 mg SNE. Antibody levels against all 9 HPV VLP types were monitored for 32 weeks. As shown in FIG. 14, the antibody concentrations (μg/mL) detected in serum against the 9 VLP types at week 32. The data are presented as geometric mean concentrations and 95% confidence intervals. These findings apply to all 9 VLP types included in the 9vHPV Vaccine.

What is claimed is:

1. A composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82 and a squalene nanoemulsion (SNE) adjuvant, wherein the SNE adjuvant comprises sorbitan trioleate (SPAN-85); polysorbate-20 (PS-20) or polysorbate-80 (PS-80); and squalene.

2. The composition of claim 1, wherein the composition is made by mixing an HPV vaccine and an SNE adjuvant; wherein the HPV vaccine comprises HPV VLPs and a pharmaceutically acceptable carrier and the SNE adjuvant comprises SPAN-85, PS-20 or PS-80, and squalene.

3. The composition of claim 1, wherein the SNE adjuvant comprises 6 μg/mL-14 mg/mL SPAN-85, 6 μg/mL-14 mg/mL PS-20 or PS-80, and 20 μg/mL-240 mg/mL of squalene.

4. The composition of claim 1, wherein the SNE comprises PS-20.

5. The composition of claim 1, wherein the composition further comprises a buffer.

6. The composition of claim 5, wherein the buffer is selected from the group consisting of: acetic acid, histidine, citrate, Bis-Tris, HEPES, phosphate, MES, sodium chloride, succinate, Tris, and combinations thereof.

7. The composition of claim 5, wherein the buffer is present in the amount of about 1 mMol to about 100 mMol.

8. The composition of claim 1, wherein the composition further comprises a salt.

9. The composition of claim 8, wherein the salt is NaCl.

10. The composition of claim 1, wherein the composition comprises 5 mM-40 mM histidine at pH 5.1-7.0 and 25 mM-300 mM NaCl.

11. The composition of claim 1, wherein the composition comprises about 20 mM histidine at about pH 5.8 and about 75 mM NaCl.

12. The composition of claim 1, wherein the composition comprises VLPs of HPV types 16 and 18.

13. The composition of claim 1, wherein the composition comprises VLPs of HPV types 6, 11, 16, and 18.

14. The composition of claim 1, wherein the composition comprises VLPs of HPV types 31, 45, 52, and 58.

15. The composition of claim 1, wherein the composition comprises VLPs of HPV types 6, 11, 16, 18, 31, 33, 45, 52, and 58.

16. The composition of claim 1, wherein the composition comprises VLPs of HPV types 6, 11, 16, 18, 31, 33, 35, 39, 45, 51, 52, 56, 58, and 59.

17. The composition of claim 1, wherein the composition comprises VLPs of HPV types 6, 11, 16, 18, 31, 33, 35, 39, 45, 51, 52, 56, 58, 59, and 73.

18. The composition of claim 1, wherein the composition comprises VLPs of HPV types 6, 11, 16, 18, 26, 31, 33, 35, 39, 45, 51, 52, 53, 56, 58, 59, 66, 68, 69, and 70.

19. The composition of claim 1, wherein the composition further comprises aluminum.

20. The composition of claim 1, wherein the HPV VLPs comprise recombinant HPV L1 or recombinant HPV L1+L2 protein.

21. The composition of claim 1, wherein the HPV VLPs comprise HPV L1 protein and do not comprise HPV L2 protein.

22. The composition of claim 1, wherein the HPV VLPs consist of HPV L1 protein.

23. A method of preventing infection of or reducing the likelihood of infection of a human patient by a human papillomavirus (HPV) comprising administration to the patient the composition of claim 1.

24. A kit comprising:
(a) a human papillomavirus (HPV) vaccine; and
(b) a squalene nanoemulsion (SNE) adjuvant, wherein the SNE adjuvant comprises sorbitan trioleate (SPAN-85); polysorbate-20 (PS-20) or polysorbate-80 (PS-80); and squalene.

25. The kit of claim 24, further comprising instructions for administering to a human patient the HPV vaccine and the SNE adjuvant.

26. The kit of claim 24, wherein the HPV vaccine comprises virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82.
